(19) United States
(12) Patent Application Publication
(10) Pub. No.: US 2005/0214283 A1
(43) Pub. Date: Sep. 29, 2005

(54) Antibodies to CD44 glycoforms and uses thereof

(76) Inventor: Robert Sackstein, Sudbury, MA (US)

Correspondence Address: FISH & RICHARDSON PC, P.O. BOX 1022, MINNEAPOLIS, MN, 55440-1022, US

(21) Appl. No.: 10/983,364

(22) Filed: Nov. 8, 2004

Related U.S. Application Data
(60) Provisional application No. 60/518,353, filed on Nov. 7, 2003.

(51) Int. Cl. A61K 39/395; C07K 16/28

(52) U.S. Cl. 424/144.1; 530/388.22

(57) ABSTRACT

Antibodies, or antigen binding portions thereof, to CD44 and specific variants and glycoforms of CD44 are provided. The anti-CD44 antibodies, or antigen binding portions thereof, modulate binding of CD44 and/or specific variants and/or glycoforms of CD44 to ligands of CD44.

### CROSS-REFERENCE TO RELATED APPLICATIONS

This application claims the benefit of priority of U.S. Ser. No. 60/518,353, filed Nov. 7, 2003, the contents of which are hereby incorporated by reference in their entirety.

### STATEMENT AS TO FEDERALLY SPONSORED RESEARCH

The work described herein was funded, in part, through a grant from the National Institutes of Health (Grant No. HL60528 awarded to Robert Sackstein). The United States government may, therefore, have certain rights in the invention.

### FIELD OF THE INVENTION

The present invention relates to antibodies that recognize various forms of the CD44 molecule and their uses in detecting CD44, and modulating the functions of CD44.

### BACKGROUND OF THE INVENTION

Tissue-specific migration of lymphocytes is critically dependent on the expression of cell membrane adhesion molecules that regulate adhesive interactions with target tissue endothelium. The selectin family of adhesion molecules consists of lectins that mediate leukocyte tethering and rolling interactions on endothelial cells. Their expression on either the leukocyte (L-selectin) or endothelium (P— and E-selectin) helps the leukocyte “slow down,” enabling cells in the blood to respond to chemokine/cytokine signals. Thus triggered, the cells firmly attach to and extravasate into a tissue site. This primary intravascular braking mechanism is controlled by Ca²⁺-dependent binding activity of the selectins to respective sialylated and fucosylated ligands expressed on leukocytes and/or endothelial cells. Selectin mediated interactions are critical not only for the rapid and efficient recruitment of leukocytes at a site of injury, but also for steady-state, tissue-specific homing as illustrated in, for example, lymphocyte homing to peripheral lymph nodes (Kansas, G. S. Blood. 88:3259-3287, 1996), cutaneous tropism of human skin-homing T cells (Picker et al. Am. J. Pathol. 136:1053-1068,1990; Berg et al. J. Exp. Med. 174:1461-1466, 1991), and hematopoietic progenitor cell (HPC) entry into bone marrow.

CD44 is a broadly distributed cell surface glycoprotein receptor for the glycosamino glycan hyaluronan (HA). Hematopoietic cell E-selectin/L-selectin ligand (HCELL) is a glycoform of CD44 containing HECA-452 reactive sialylated, fucosylated N-glycans. HCELL is a ligand for both L-selectin and E-selectin (Dimitroff et al. Proc. Natl. Acad. Sci. USA, 97(25):13841-46, 2000; Dimitroff et al., J. Cell Biol., 153(6): 1277-1286, 2001). CD44 is expressed on a diverse variety of cell types including most hematopoietic cells, keratinocytes, chondrocytes, many epithelial cell types, and some endothelial and neural cells. CD44 is known to participate in a wide variety of cellular functions, including cell-cell aggregation, retention of pericellular matrix, matrix-cell and cell-matrix signaling, receptor-mediated internalization/degradation of hyaluronan, and cell migration. Various functions of CD44 are dependent upon adhesive interactions with hyaluronan. Some CD44 functions are affected or induced by post-translational modifications in the CD44 protein such as sulfation and/or glycosylations.

### SUMMARY OF THE INVENTION

The invention is based, in part, on the discovery of antibodies that can modulate one or more functions of CD44 molecules. CD44 molecules interact with various ligands to mediate adhesive interactions of leukocytes. For example, CD44 interacts with hyaluronan. The HCELL glycoform of CD44 interacts with L-selectin and E-selectin. The discovered antibodies can increase or decrease binding of CD44 to one or more of CD44 ligands such as hyaluronan, E-selectin, and L-selectin. In some cases, the antibody increases binding of CD44 (e.g., a glycoform of CD44, e.g., HCELL) to one ligand and decreases binding of CD44 (e.g., a glycoform of CD44, e.g., HCELL) to another ligand.

This invention provides, inter alia, antibodies or antigen binding portions thereof that bind to CD44, including specific variants of CD44, such as an HCELL glycoform of CD44. The anti-CD44 antibodies, or portions thereof, bind to specific variants of human CD44 with high affinity and specificity, and thus can be used as diagnostic, prophylactic, or therapeutic agents in vivo and in vitro. The anti-CD44 antibodies, or portions thereof, modulate activities of CD44 and/or activities of specific glycoforms of CD44, e.g., an HCELL glycoform of CD44. Accordingly, the invention provides antibodies and pharmaceutical compositions thereof, as well as nucleic acids, recombinant expression vectors and host cells for making such antibodies and portions. Methods of using the antibodies of the invention to detect CD44 and/or a specific glycoform of CD44, e.g., HCELL, or to select or ablate a CD44-expressing cell, e.g., a hematopoietic cell, an HCELL-expressing cell, or a cancer, either in vitro or in vivo, are also encompassed by the invention. As discussed herein, the antibodies can be “humanized” or modified in a variety or ways (e.g., alterations in the Fc portion or in other structural features such as to improve the function, stability, bioavailability or spectrum of activity of the antibody). The antibodies can be derived in vitro (e.g., using phage display technology).

The antibodies or antigen binding portions thereof bind to one or more specific forms of CD44, preferably human CD44, e.g., a glycoform of CD44, e.g., a human HCELL, with high affinity and specificity. For example, in one embodiment, the antibody or antigen binding portion thereof binds to one or more specific forms of human CD44 with an affinity constant of at least 10⁷ M⁻¹, preferably between 10⁸ M⁻¹ and 10¹⁰ M⁻¹, or about 10⁹ M⁻¹. Preferably, the antibody or antigen binding portion thereof modulates an activity of CD44 and/or of a glycoform of CD44. For example, the antibody or antigen binding portion thereof modulates an interaction of CD44 with a ligand, e.g., the antibody inhibits or agonizes binding of CD44 to hyaluronan, E-selectin and/or L-selectin. Preferably, the antibody interacts with, e.g., binds to, the extracellular domain of CD44, e.g., an extracellular domain of one or more specific forms of CD44.

Human CD44 is expressed on the surface of certain mature leukocytes. For example, HCELL is expressed on CD34⁺ subsets of human hematopoietic cells, e.g., hematopoietic stem cells, as well as embryonic stem cells, and cancer stem cells. The antibodies of the invention bind to the surface of cells that express CD44. Accordingly, the antibodies or antigen binding portions thereof described herein, can be used to target living cells expressing CD44 and/or specific glycoforms of CD44, such as HCELL.

“HCELL” refers to a CD44 molecule comprising glycosylation(s) that render it an E-selectin and/or L-selectin ligand. An HCELL may have L-selectin binding activity, E-selectin binding activity, or both.

Accordingly, the invention features an isolated antibody or antigen binding portion thereof that binds to CD44, including a glycoform of CD44 that has E-selectin and/or L-selectin ligand activity such as HCELL, wherein the antibody or antigen binding portion thereof includes one or more of the following characteristics: (a) the antibody or antigen binding portion thereof specifically binds to C44 including glycosylations of CD44 that render selectin ligand activity; (b) the antibody or antigen binding portion thereof modulates binding of CD44 or HCELL to hyaluronan; (c) the antibody or antigen binding portion thereof modulates binding of HCELL to E-selectin; (d) the antibody or antigen binding portion thereof modulates binding of HCELL to L-selectin.

In one embodiment, the antibody or antigen binding portion modulates binding of CD44 to hyaluronan. In one embodiment, the antibody or antigen binding portion thereof agonizes binding of CD44 to hyaluronan. In one embodiment, the antibody or antigen binding portion thereof inhibits binding of HCELL to hyaluronan. In another embodiment, the antibody or antigen binding portion thereof modulates binding of HCELL to E-selectin. In one embodiment, the antibody or antigen binding portion thereof agonizes binding of HCELL to E-selectin. In one embodiment, the antibody or antigen binding portion thereof inhibits binding of HCELL to E-selectin.

In another embodiment, the antibody or antigen binding portion thereof modulates binding of HCELL to L-selectin. In one embodiment, the antibody or antigen binding portion thereof agonizes binding of HCELL to L-selectin. In one embodiment, the antibody or antigen binding portion thereof inhibits binding of HCELL to L-selectin.

In one embodiment, the antibody or antigen binding portion thereof modulates binding of HCELL to hyaluronan and modulates binding of HCELL to E-selectin. In one embodiment, the antibody or antigen binding portion thereof agonizes binding of HCELL to hyaluronan and agonizes binding of HCELL to E-selectin. In one embodiment, the antibody or antigen binding portion thereof agonizes binding of HCELL to hyaluronan and inhibits binding of HCELL to E-selectin. In one embodiment, the antibody or antigen binding portion thereof inhibits binding of HCELL to hyaluronan and inhibits binding of HCELL to E-selectin. In one embodiment, the antibody or antigen binding portion thereof inhibits binding of HCELL to hyaluronan and agonizes binding of HCELL to E-selectin. In one embodiment, the antibody or antigen binding portion thereof does not specifically bind to a non-HCELL glycoform of CD44.

In another embodiment, the antibody or antigen binding portion thereof modulates binding of HCELL to hyaluronan and modulates binding of HCELL to L-selectin. In one embodiment, the antibody or antigen binding portion thereof agonizes binding of HCELL to hyaluronan and agonizes binding of HCELL to L-selectin. In one embodiment, the antibody or antigen binding portion thereof agonizes binding of HCELL to hyaluronan and inhibits binding of HCELL to L-selectin. In one embodiment, the antibody or antigen binding portion thereof inhibits binding of HCELL to hyaluronan and inhibits binding of HCELL to L-selectin. In one embodiment, the antibody or antigen binding portion thereof inhibits binding of HCELL to hyaluronan and agonizes binding of HCELL to L-selectin. In one embodiment, the antibody or antigen binding portion thereof does not specifically bind to a non-HCELL glycoform of CD44.

In another embodiment, the antibody or antigen binding portion thereof modulates binding of HCELL to E-selectin and modulates binding of HCELL to L-selectin. In one embodiment, the antibody or antigen binding portion thereof agonizes binding of HCELL to E-selectin and agonizes binding of HCELL to L-selectin. In one embodiment, the antibody or antigen binding portion thereof agonizes binding of HCELL to E-selectin and inhibits binding of HCELL to L-selectin. In one embodiment, the antibody or antigen binding portion thereof inhibits binding of HCELL to E-selectin and agonizes binding of HCELL to L-selectin. In one embodiment, the antibody or antigen binding portion thereof inhibits binding of HCELL to E-selectin and to L-selectin. In one embodiment, the antibody or antigen binding portion thereof does not specifically bind to a non-HCELL glycoform of CD44.

In one embodiment, the antibody or antigen binding portion thereof modulates binding of HCELL to hyaluronan, modulates binding of HCELL to E-selectin, and modulates binding of HCELL to L-selectin. In one embodiment, the antibody or antigen binding portion thereof agonizes binding of HCELL to hyaluronan, E-selectin, and L-selectin. In one embodiment, the antibody or antigen binding portion thereof agonizes binding of HCELL to hyaluronan and E-selectin, and inhibits binding of HCELL to L-selectin. In one embodiment, the antibody or antigen binding portion thereof agonizes binding of HCELL to hyaluronan, and inhibits binding of HCELL to E-selectin and L-selectin. In one embodiment, the antibody or antigen binding portion thereof inhibits binding of HCELL to hyaluronan, E-selectin, and L-selectin. In one embodiment, the antibody or antigen binding portion thereof inhibits binding of HCELL to hyaluronan and E-selectin, and agonizes binding of HCELL to L-selectin. In one embodiment, the antibody or antigen binding portion thereof inhibits binding of HCELL to hyaluronan, and agonizes binding of HCELL to E-selectin and L-selectin. In one embodiment, the antibody or antigen binding portion thereof inhibits binding of HCELL to hyaluronan and L-selectin and agonizes binding of HCELL to E-selectin. In one embodiment, the antibody or antigen binding portion thereof agonizes binding of HCELL to hyaluronan and L-selectin and inhibits binding of HCELL to E-selectin. In one embodiment, the antibody or antigen binding portion thereof does not specifically bind to a non-HCELL glycoform of CD44.

In one embodiment, the antibody or antigen binding portion thereof binds HCELL with an affinity of at least 10⁶ M⁻¹, preferably at least 10⁷ M⁻¹, 10⁸ M⁻¹, or 10¹⁰ M⁻¹.

In one embodiment, the antibody or antigen binding portion thereof inhibits binding of HCELL to any of its ligands (e.g., hyaluronan, L-selectin or E-selectin) by at least 10% relative to a control.

In one embodiment, the antibody or antigen binding portion thereof agonizes binding of HCELL to any of its ligands (e.g., hyaluronan, L-selectin or E-selectin) by at least 10% relative to a control (e.g., HCELL binding in the absence of the antibody).

In one embodiment, the antibody or antigen binding portion thereof does not compete for binding to an epitope bound by HECA-452 on CD44. In an another embodiment, the antibody competes for binding to an epitope bound by HECA-452.

In one embodiment, the antibody or antigen binding portion binds a saccharide epitope of CD44. In one embodiment, the antibody or antigen binding portion thereof binds a selectin-binding saccharide epitope of CD44 (e.g., an HCELL-specific saccharide epitope). In one embodiment, the antibody or antigen binding portion thereof binds a protein epitope of CD44. In one embodiment, the antibody or antigen binding portion thereof binds a protein and a saccharide epitope of CD44.

The antibody or antigen binding portion thereof can be monoclonal or polyclonal. In one embodiment, the antibody is a murine monoclonal antibody. In one embodiment, the antibody is humanized. In one embodiment, the antibody is chimeric, deimmunized, or human. In one embodiment, the antibody is an IgG or is derived from an IgG In one embodiment, the antibody further includes a label. In one embodiment, the antibody includes a toxin. In one embodiment, the antibody can mediate complement mediated cytotoxicity or antigen-dependent cellular cytotoxicity.

In another aspect, the invention features an isolated antibody or antigen binding portion thereof that specifically binds to an HCELL glycoprotein, wherein the antibody or antigen binding portion thereof binds a denatured form of HCELL (e.g., SDS-denatured HCELL, e.g., SDS-denatured HCELL bound to nitrocellulose for in a Western blot). In one embodiment, the antibody or antigen binding portion thereof also specifically binds a native form of HCELL (e.g., expressed on the surface of a HCELL-expressing (HCELL⁺) cell, e.g., a KG1a cell, a primitive adult stem cell, such as a CD34⁺ hematopoietic stem cell, an embryonic stem cell, a cancer stem cell). In one embodiment, the antibody or antigen binding portion thereof does not specifically bind a non-HCELL glycoform of CD44. In one embodiment, the antibody binds HCELL with an affinity of at least 10⁶M⁻¹.

The antibody or antigen binding portion thereof can be monoclonal or polyclonal. In one embodiment, the antibody is a murine monoclonal antibody. In one embodiment, the antibody is humanized. In one embodiment, the antibody is chimeric, deimmunized, or human. In one embodiment, the antibody is an IgG or is derived from an IgG. In one embodiment, the antibody further includes a label. In one embodiment, the antibody includes a toxin. In one embodiment, the antibody can mediate complement mediated cytotoxicity or antigen-dependent cellular cytotoxicity.

In another aspect, the invention features an isolated antibody or antigen binding portion thereof that specifically binds to CD44, wherein the antibody or antigen binding portion thereof agonizes binding of CD44 to hyaluronan. In one embodiment, the antibody or antigen binding portion thereof agonizes binding of CD44 to hyaluronan by at least 2, 5, or 10-fold. In one embodiment, the antibody or antigen binding portion thereof does not specifically bind an HCELL glycoform of CD44. In one embodiment, the antibody or antigen binding portion thereof binds CD44 with an affinity of at least 10⁶ M⁻¹. The antibody or antigen binding portion thereof can be monoclonal or polyclonal. In one embodiment, the antibody is a murine monoclonal antibody. In one embodiment, the antibody is humanized. In one embodiment, the antibody is chimeric, deimmunized, or human. In one embodiment, the antibody is an IgG or is derived from an IgG. In one embodiment, the antibody further includes a label. In one embodiment, the antibody includes a toxin. In one embodiment, the antibody can mediate complement mediated cytotoxicity or antigen-dependent cellular cytotoxicity.

In another aspect, the invention features an isolated antibody or antigen binding portion thereof that specifically binds to CD44, wherein the antibody or antigen binding portion thereof inhibits binding of CD44 to hyaluronan. In another embodiment, the antibody enhances binding to hyaluronic acid.

In one embodiment, the antibody or antigen binding portion thereof inhibits binding by at least 10%, 15%, 20%, 40%, 50%, 60%, 70%, 80%, 90% relative to a control. In one embodiment, the antibody or antigen binding portion thereof binds CD44 with an affinity of at least 10⁻⁶ M. In one embodiment, the antibody or antigen binding portion thereof inhibits binding of CD44 to L-selectin. In one embodiment, the antibody or antigen binding portion thereof inhibits binding of CD44 to E-selectin. In one embodiment, the antibody or antigen binding portion thereof agonizes binding of CD44 to L-selectin. In one embodiment, the antibody or antigen binding portion thereof agonizes binding of CD44 to E-selectin.

The antibody or antigen binding portion thereof can be monoclonal or polyclonal. In one embodiment, the antibody is a murine monoclonal antibody. In one embodiment, the antibody is humanized. In one embodiment, the antibody is chimeric, deimmunized, or human. In one embodiment, the antibody is an IgG or is derived from an IgG. In one embodiment, the antibody further includes a label. In one embodiment, the antibody includes a toxin. In one embodiment, the antibody can mediate complement mediated cytotoxicity or antigen-dependent cellular cytotoxicity.

In another aspect, the invention features an isolated antibody or antigen binding portion thereof that specifically binds to an HCELL glycoprotein, wherein the antibody is produced by immunizing any animal (e.g., rat, rabbit, hamster, chicken, etc.) with a sample including an HCELL glycoprotein and isolating an antibody or antigen binding portion thereof that specifically binds HCELL from the animal. In one embodiment, the sample includes a cell that expresses HCELL. In one embodiment, the sample includes a human stem cell such as a CD34⁺ cell that expresses HCELL. In one embodiment, the sample includes a KG1a cell, or a cell of a subline of KG1a cells. In one embodiment, the sample includes HCELL obtained from a KG1a cell.

In another aspect, the invention features an isolated antibody or antigen binding portion thereof that specifically binds to CD44, wherein the antibody is made by a method which includes, for example: purifying HCELL from a KG1a cell; injecting the purified HCELL into a mouse; obtaining a monoclonal antibody from the mouse which binds HCELL. In one embodiment, the method further includes obtaining the monoclonal antibody by one or more of: generating a set of monoclonal antibodies from the mouse; screening the set of monoclonal antibodies for binding to a KG1a cell; identifying antibodies of the set that bind a KG1a cell; screening the identified antibodies for binding to a cell expressing CD44; and selecting an antibody that binds the CD44⁺ cell. In one embodiment, the method further includes determining if the identified antibodies bind to intact or denatured CD44 (e.g., as in a Western blot). In one embodiment, the method further includes selecting an antibody that binds intact or denatured CD44. In one embodiment, the method further includes the steps of: evaluating modulation of interaction of CD44 with one or more ligand of CD44 such as hyaluronan, E-selectin, and L-selectin by the antibody.

In another aspect, the invention features an isolated antibody or antigen binding portion thereof that specifically binds to an HCELL glycoprotein, wherein the antibody or antigen binding portion thereof includes the following characteristics: (a) the antibody specifically binds to a cell of the KG1a cell line or a human stem cell such as a CD34+ cell or a cancer stem cell (e.g., leukemia); (b) the antibody does not substantially bind cells of the following hematopoietic stem cell lines: Jurkat and K562 erythrocytic leukemia cells.

The antibody or antigen binding portion thereof can be monoclonal or polyclonal. In one embodiment, the antibody is a murine monoclonal antibody. In one embodiment, the antibody is humanized. In one embodiment, the antibody is chimeric, deimmunized, or human. In one embodiment, the antibody is an IgG or is derived from an IgG. In one embodiment, the antibody further includes a label. In one embodiment, the antibody includes a toxin. In one embodiment, the antibody can mediate complement mediated cytotoxicity or antigen-dependent cellular cytotoxicity.

In another aspect, the invention features an isolated antibody or antigen binding portion thereof that specifically binds to an HCELL glycoprotein, wherein the antibody or antigen binding portion thereof enhances engraftment of hematopoietic stem cells in vivo.

The antibody or antigen binding portion thereof can be monoclonal or polyclonal. In one embodiment, the antibody is a murine monoclonal antibody. In one embodiment, the antibody is humanized. In one embodiment, the antibody is chimeric, deimmunized, or human. In one embodiment, the antibody is an IgG or is derived from an IgG. In one embodiment, the antibody further includes a label. In one embodiment, the antibody includes a toxin. In one embodiment, the antibody can mediate complement mediated cytotoxicity or antigen-dependent cellular cytotoxicity.

In one aspect, the invention features an antibody or antigen binding portion thereof that specifically binds a human HCELL glycoprotein.

The antibody or antigen binding portion thereof can be monoclonal or polyclonal. In one embodiment, the antibody is a murine monoclonal antibody. In one embodiment, the antibody is humanized. In one embodiment, the antibody is chimeric, deimmunized, or human. In one embodiment, the antibody is an IgG or is derived from an IgG.

In one embodiment, the antibody further includes a label. In one embodiment, the antibody includes a toxin. In one embodiment, the antibody can mediate complement mediated cytotoxicity or antigen-dependent cellular cytotoxicity.

An anti CD44 antibody can be monospecific, e.g., a monoclonal antibody, or an antigen binding portion thereof. The antibodies can be full-length (e.g., an IgG (e.g., an IgG1, IgG2, IgG3, IgG4), IgM, IgA (e.g., IgA1, IgA2), IgD, and IgE, but preferably an IgG) or can include only an antigen binding portion (e.g., a Fab, F(ab′)₂ or scFv fragment, or one or more CDRs). An antibody, or antigen binding portion thereof, can include two heavy chain immunoglobulins and two light chain immunoglobulins, or can be a single chain antibody. The antibodies can, optionally, include a constant region chosen from a kappa, lambda, alpha, gamma, delta, epsilon or a mu constant region gene. In one embodiment, the anti-CD44 antibody includes a heavy and light chain constant region substantially from a human antibody, e.g., a human IgG1 constant region, a portion thereof, or a consensus sequence.

Any combination of anti-CD44 antibodies is within the scope of the invention, e.g., two or more antibodies that bind to different regions of CD44, e.g., antibodies that bind to two different epitopes on the extracellular domain of CD44.

In some embodiments, the anti-CD44 antibody, or antigen binding portion thereof, includes at least one light or heavy chain immuglobulin (or preferably, at least one light chain immuglobulin and at least one heavy chain immuglobulin). Preferably, each immunoglobulin includes a light or a heavy chain variable region having at least one, two and, preferably, three CDRs substantially identical to a CDR from a non-human or a human anti-CD44 light or heavy chain variable region, respectively. In one preferred embodiment, the antibody or antigen binding portion thereof includes all six CDRs from the same non-human or human anti-CD44 antibody.

The light or heavy chain immunoglobulin of the anti-CD44 antibody or antigen binding portion thereof can further include a light chain or a heavy chain variable framework sequence from a light chain or heavy chain variable framework present in a human or a non-human, e.g., rodent, antibody.

An anti-CD44 antibody, or antigen binding portion thereof, described herein can be used alone, e.g., can be administered to a subject, or used in vitro, in non-derivatized or unconjugated forms. In other embodiments, the anti-CD44 antibody, or antigen binding portion thereof, can be derivatized or linked to another molecular entity, typically a label or a therapeutic (e.g., a cytotoxic or cytostatic) agent. The molecular entity can be, e.g., another peptide, protein (including, e.g., a viral coat protein of, e.g., a recombinant viral particle), a non-peptide chemical compound, isotope, etc. The anti-CD44 antibody, or antigen binding portion thereof, can be functionally linked, e.g., by chemical coupling, genetic fusion, non-covalent association or otherwise, to one or more other molecular entities. For example, the anti-CD44 antibody, or antigen binding portion thereof, can be coupled to a label, such as a fluorescent label, a biologically active enzyme label, a radioisotope (e.g., a radioactive ion), a nuclear magnetic resonance active label, a luminescent label, or a chromophore. In other embodiments, the anti-CD44 antibody, or antigen binding portion thereof, can be coupled to a therapeutic agent, e.g., a cytotoxic moiety, e.g., a therapeutic drug, a radioisotope, molecules of plant, fungal, or bacterial origin, or biological proteins (e.g., protein toxins) or particles (e.g., recombinant viral particles, e.g., via a viral coat protein), or mixtures thereof. The therapeutic agent can be an intracellularly active drug or other agent, such as short-range radiation emitters, including, for example, short-range, high-energy α-emitters, as described herein. In some preferred embodiments, the anti-CD44 antibody, or antigen binding portion thereof, can be coupled to a molecule of plant or bacterial origin (or derivative thereof). A radioisotope can be an α-, β-, or γ-emitter, or an β- and γ-emitter. Radioisotopes useful as therapeutic agents include yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), and rhodium (¹⁸⁸Rh). Radioisotopes useful as labels, e.g., for use in diagnostics, include iodine (¹³¹I or ¹²⁵I), indium (¹¹¹In), technetium (⁹⁹mTc), phosphorus (³²p), carbon (¹⁴C), and tritium (³H), or one of the therapeutic isotopes listed above. The anti-CD44 antibody, or antigen binding portion thereof can be linked to an anti-inflammatory agent or an immunosuppressive agent. The anti-CD44 antibody, or antigen binding portion thereof can also be linked to another antibody to form, e.g., a bispecific or a multispecific antibody.

In another aspect, the invention features a method for making an anti-HCELL antibody. The method includes, for example: immunizing an animal with a sample including an HCELL glycoprotein, and isolating an antibody that specifically binds HCELL from the animal. The method can further include evaluating modulation of an HCELL interaction with one or more of hyaluronan, E-selectin, and L-selectin by the antibody. The sample is, e.g., a cell expressing HCELL, or a membrane preparation from a cell expressing HCELL.

In yet another aspect, the invention features a method for making an anti-HCELL antibody, wherein the method includes, for example: purifying HCELL from an HCELL-expressing cell (e.g., a KG1a cell); injecting the purified HCELL into an animal; generating a set of antibodies from cells from the animal; and isolating a monoclonal antibody of the set that binds HCELL.

In one embodiment, the invention features a method of identifying an antibody that specifically binds to CD44, the method including: providing a CD44 antigen; providing a phage display library that expresses antibodies; identifying a member present in the library that specifically binds to the CD44 antigen; and isolating a nucleic acid molecule from the identified member, wherein the nucleic acid molecule encodes the antibody that specifically binds to the CD44 antigen. The method can further include the step of expressing the antibody in a mammalian cell; and evaluating modulation of interactions of CD44 with CD44 ligands by the antibody. In one embodiment, modulation (e.g., antagonism, agonism) of CD44-hyaluronan interactions is evaluated. In one embodiment, modulation (e.g., antagonism, agonism) of CD44-E-selectin interactions is evaluated. In one embodiment, modulation (e.g., antagonism, agonism) of CD44-L-selectin interactions is evaluated.

In one aspect, the invention features a method of evaluating an antibody or antigen binding portion thereof for binding to HCELL. The method includes, for example: providing an HCELL antigen, contacting the sample with an antibody or antigen binding portion thereof; and evaluating binding of the antibody or antigen binding portion thereof to the HCELL antigen. The method can further include evaluating interaction of HCELL with hyaluronan, E-selectin and/or L-selectin.

In another aspect, the invention features a method of detecting HCELL in a sample. The method includes, for example: providing a sample; contacting the sample with an anti-HCELL antibody or antigen binding portion thereof as described herein; and detecting the anti-HCELL antibody or antigen binding portion thereof. In one embodiment, the anti-HCELL antibody or antigen binding portion thereof is labeled (e.g., labeled with a fluorescent label, a biologically active enzyme label, a radioisotope, a luminescent label, or a chromophore).

In another aspect, the invention features a method of modulating an activity of an HCELL-expressing cell. The method includes, for example: contacting an HCELL-expressing cell with an antibody or antigen binding portion thereof, e.g., an antibody or antigen binding portion thereof described herein in an amount sufficient to modulate an activity of the cell, thereby modulating the activity of the HCELL-expressing cell. In one embodiment, the modulating is agonizing the activity. In one embodiment, the modulating is inhibiting the activity. In one embodiment, the activity is binding to a selectin. In one embodiment, the activity is binding to E-selectin. In one embodiment, the activity is binding to L-selectin. In one embodiment, the activity is binding to hyaluronan.

In one embodiment, the anti-CD44 antibody inhibits interaction of CD44 with a CD44 ligand. In one embodiment, the anti-CD44 antibody agonizes, or increases interaction of CD44 with a CD44 ligand. Antibodies that inhibit an activity of CD44 (e.g., a binding interaction between CD44 and a CD44 ligand) can be used, e.g., to inhibit inflammatory and/or autoimmune reactions mediated by leukocytes, thereby treating inflammatory and/or autoimmune diseases.

The methods can be used on cells in culture, e.g. in vitro or ex vivo. For example, cells can be cultured in vitro in culture medium and the contacting step can be effected by adding the anti-CD44 antibody or portion thereof, to the culture medium. Cells treated ex vivo can be re-infused into a subject following the contacting step. The method can be performed on cells present in a subject, as part of an in vivo (e.g., therapeutic or prophylactic) protocol.

In another aspect, the invention features a method of modulating two or more activities of an HCELL-expressing cell. The method includes, for example: contacting an HCELL-expressing cell with an antibody or antigen binding portion thereof (e.g., an antibody or antigen binding portion thereof as described herein), thereby modulating the activities of the HCELL-expressing cell. In one embodiment, the modulating includes agonizing an activity. In one embodiment, the modulating includes inhibiting an activity. In one embodiment, the modulating includes agonizing an activity and inhibiting an activity. Activities include: binding to E-selectin, L-selectin, and/or hyaluronan.

In another aspect, the invention features a method of modulating HCELL activity in a subject. The method includes, for example: identifying a subject in need of modulated HCELL activity; and administering the antibody or antigen binding portion thereof (e.g., an antibody or antigen binding portion thereof as described herein) to the subject.

In another aspect, the invention features a method for isolating a subset of hematopoietic cells. The method includes, for example: providing a population of cells, wherein the population includes hematopoietic cells; contacting the cells with an anti-HCELL antibody or antigen binding portion thereof (e.g., an antibody or antigen binding portion thereof as described herein) under conditions that allow the anti-HCELL antibody or antigen binding portion thereof to bind the HCELL-expressing cells; removing the cells that do not bind the anti-HCELL antibody or antigen binding portion thereof, thereby isolating a subset of hematopoietic cells. In one embodiment, the antibody or antigen binding portion thereof does not bind one or more non-HCELL forms of CD44.

In another aspect, the invention features a method for enriching a population of hematopoietic cells, e.g., for cells that have blood-forming capacity. The method includes, for example: providing a population of hematopoietic cells; contacting the cells with an anti-HCELL antibody or antigen binding portion thereof (e.g., an antibody or antigen binding portion thereof as described herein) under conditions that allow the anti-HCELL antibody or antigen binding portion thereof to bind the HCELL-expressing cells; removing the cells that do not bind the anti-HCELL antibody or antigen binding portion thereof, thereby enriching a population of hematopoietic cells for cells that have functional activity such as blood-forming capacity. In one embodiment, the antibody or antigen binding portion thereof only binds the HCELL form of CD44. In one embodiment, the anti-HCELL antibody or antigen binding portion thereof is immobilized on the solid phase or a bead (e.g., a magnetic bead) In one embodiment, the method further includes recovering the cells that bind the anti-HCELL antibody or portion thereof. The method can further include administering the cells to a subject. The cells can be stored (e.g., by freezing) prior to administration to the subject.

In another aspect, the invention features a method for enhancing engraftment of hematopoietic stem cells in a subject. The method includes, for example: administering to the subject an anti-HCELL antibody or antigen binding portion thereof (e.g., an antibody or antigen binding portion thereof as described herein), wherein the antibody or antigen binding portion thereof agonizes HCELL-selectin interactions, thereby enhancing engraftment of hematopoietic stem cells in the subject (e.g., to treat patients undergoing hematopoietic stem cell transplantation for cancer or other hematopoietic disorders). In one embodiment, the antibody or antigen portion thereof agonizes HCELL-selectin interactions (e.g., HCELL-E-selectin interactions, and/or HCELL-L-selectin interactions). In one embodiment, the anti-HCELL antibody or antigen binding portion thereof enhances binding of CD44 to hyaluronic acid or to other CD44 ligands (e.g., fibronectin); in these cases, the antibody or antigen binding portion thereof may or may not recognize the HCELL glycoform of CD44. Antibodies can be administered by vascular access (e.g., IV), intramuscularly, subcutaneously or within body tissues/cavities.

In another aspect, the invention features a hematopoietic stem cell isolated by a method, the method including: providing a population of hematopoietic cells; contacting the cells with an anti-HCELL antibody or antigen binding portion thereof (e.g., an antibody or antigen binding portion thereof as described herein), under conditions that allow the anti-HCELL antibody or antigen binding portion thereof to bind the HCELL-expressing cells; removing the cells that do not bind the anti-HCELL antibody or antigen binding portion thereof, isolating a cell that binds the anti-HCELL antibody or antigen binding portion thereof.

The antibodies and antigen binding portions described herein can be used in methods of treating or preventing disorders, including, e.g., disorders characterized by HCELL-selectin interactions, and disorders characterized by HCELL/CD44-hyaluronan interactions. These disorders include, e.g., hematopoietic disorders, cancers, cancer-related disorders, inflammatory disorders, autoimmune disorders, vascular diseases (ischemic-reperfusion injury, atherosclerosis), and fertility disorders. The methods include administering to a subject an antibody or antigen binding portion thereof described herein, preferably an anti-CD44 antibody or antigen binding portion thereof, in an amount effective to treat or prevent such disorder.

Examples of hematopoietic disorders that can be treated or prevented include, but are not limited to, hematopoietic disorders (e.g., aplastic anemia), including hematopoietic cancer related disorders (e.g., in the treatment of hematopoietic cancers, e.g., for hematopoietic stem cell transplantation associated with hematopoietic cancers, and for stem cell transplantation associated with non-hematopoietic cancers), anemia, neutropenia, and other bone marrow failure states.

Examples of inflammatory and autoimmune disorders that can be treated or prevented include, but are not limited to dermatitis (e.g., contact dermatitis, atopic dermatitis), cutaneous infection (e.g., bacterial, viral, fungal, or parasitic infections of the skin), pemphigus vulgaris, graft versus host disease (e.g., acute cutaneous graft versus host disease), psoriasis, drug-related hypersensitivity reactions, ultraviolet light induced injury, and acne. Other inflammatory disorders that can be treated according to the methods described herein include pulmonary disorders such as acute lung injury, acute respiratory distress syndrome, asthma, bronchitis, and cystic fibrosis; inflammatory disorders of joints, such as arthritis (e.g., rheumatoid arthritis); inflammatory disorders of the gut, such as inflammatory bowel disease, Crohn's disease and ulcerative colitis; inflammatory disorders of the nervous system, such as encephalomyelitis, and multiple sclerosis; inflammatory disorders of the endocrine system (e.g., Grave's disease, Hashimoto's thyroiditis); multiorgan inflammatory disorders (e.g., systemic lupus erythematosus); and renal, vascular, and hepatic disorders such as hepatitis (e.g., viral chronic hepatitis), reperfusion injury (e.g., myocardial), nephritis, pancreatitis, artery occlusion (e.g., retinal), and/or vasculitis. The inflammatory disease may be acute or chronic, and is preferably mediated by leukocytes. In some embodiments, the inflammatory disease is associated with chronic inflammation, e.g., the disease is inflammatory bowel disease, such as, psoriasis, sarcoidosis and rheumatoid arthritis. In one embodiment, the disorder is arthritis.

In one embodiment, the antibodies and antigen binding portions thereof described herein are used in methods of administering stem cells (e.g., stem cells with capacity to regenerate neuronal, hepatic, renal, cardiac, muscular, and other tissues) to treat disorders in which tissue regeneration is desired (e.g., stroke, diabetes, liver disease, pulmonary disease, burns).

Examples of cancers that can be treated or prevented include, but are not limited to myelodysplastic syndromes cutaneous cancers such as melanomas, basal cell and squamous cell carcinomas, Kaposi's sarcoma, leukemias, and lymphomas (e.g., chronic myeloid leukemia, chronic lymphatic leukemia, chronic granulocytic leukemia, acute lymphocytic leukemia, acute myeloid leukemia, acute myelocytic leukemia, Hodgkin's disease, non-hodgkin's lymphomas, Burkitt's lymphomas, and mycosis fungoides). Also included are neoplastic disorders and disorders of solid tumors such as adenocarcinomas of the breast, lung, kidney, uterus, prostate, bladder, ovary, colon; sarcomas such as liposarcoma, synovial sarcoma, rhabdomyosarcoma, Ewing's tumor, neuroepithelioma; and other tumors such as retinoblastoma, Wilm's tumor, and mesothelioma. In one embodiment, cancer-related disorder is a hematopoietic cancer, e.g., acute lymphocytic leukemia or acute myeloid leukemia. In one embodiment, the disorder is a cancer and the cancer is an HCELL-expressing cancer.

In one embodiment the subject is treated to prevent a disorder, e.g., a hematopoietic disorder, a cancer-related disorder, a cancer, an inflammatory disorder, an autoimmune disorder, or a fertility disorder. The subject can be one at risk for the disorder, e.g., a subject having a relative afflicted with the disorder, e.g., a subject with one or more of a grandparent, parent, uncle or aunt, sibling, or child who has or had the disorder, or a subject having a genetic trait associated with risk for the disorder. The subject can be a mammal, e.g., a primate, preferably a higher primate, e.g., a human (e.g., a patient having, or at risk of, a disorder described herein). The anti-CD44 antibody or portion thereof can be administered to the subject systemically (e.g., orally, parenterally, subcutaneously, intravenously, rectally, intramuscularly, intraperitoneally, intranasally, transdermally, or by inhalation or intracavitary installation), topically, or by application to mucous membranes, such as the nose, throat and bronchial tubes.

In one aspect, the invention features a method of treating a disorder characterized by HCELL-selectin interactions. The method includes, for example: administering to a subject an antibody or antigen binding portion thereof (e.g., an antibody or antigen binding portion thereof as described herein) that modulates an HCELL-selectin interaction, or antigen binding portion thereof, to thereby treat the disorder. In one embodiment, the antibody or antigen binding portion thereof agonizes one or more HCELL-selectin interactions. In one embodiment, the antibody or antigen binding portion thereof inhibits one or more HCELL-selectin interactions. In one embodiment, the disorder is a hematopoietic disorder, an inflammatory or autoimmune disorder, or a cancer-related disorder, atherosclerosis and other vascular diseases.

In one embodiment, the disorder is a hematopoietic disorder. In one embodiment, the disorder is an inflammatory or autoimmune disorder, e.g., a cutaneous inflammatory or autoimmune disorder, e.g., acute cutaneous graft versus host disease, psoriasis, atopic dermatitis or contact dermatitis, and burn injuries.

In one embodiment, the disorder is a cancer, e.g., a cancer or the breast, colon, or ovaries.

In another aspect, the invention feature a method for treating a disorder characterized by HCELL/CD44-hyaluronan interactions. The method includes, for example: administering to a subject an antibody or antigen binding portion thereof (e.g., an antibody or antigen binding portion thereof described herein) that modulates an HCELL/CD44-hyaluronan interaction, to thereby treat the disorder. In one embodiment, the antibody or antigen binding portion thereof agonizes an HCELL/CD44-hyaluronan interaction.

In one embodiment, the antibody or antigen binding portion thereof inhibits an HCELL/CD44-hyaluronan interaction. In one embodiment, the disorder is a hematopoietic disorder, an inflammatory disorder, a cancer-related disorder, or an autoimmune disorder.

In one embodiment, the inflammatory disorder is arthritis. In one embodiment, the disorder is graft-versus-host disease.

In one embodiment, the antibody or antigen binding portion thereof agonizes an HCELL/CD44-hyaluronan interaction. In one embodiment, the disorder is a disorder in which an enhanced immune response is desired, e.g., an infection, an anti-tumor response. In one embodiment, the disorder is a hematopoietic disorder in which hematopoietic stem cell transplantation is indicated. In one embodiment, the disorder is a disorder in which tissue regeneration is indicated (e.g., a neurodegenerative disease, stroke, heart disease, pulmonary disease, wound healing).

In another aspect, the invention features a method for characterizing a hematopoietic cancer. The method includes, for example: evaluating binding of an anti-HCELL antibody or antigen binding portion thereof (e.g., an antibody or antigen binding portion thereof as described herein) to the cancer, thereby characterizing the cancer. In one embodiment, the cancer is a hematopoietic cancer (e.g., a lymphoma, or a leukemia).

The invention also features a method of ablating or killing, a cell, e.g., a cancer cell, or a leukocyte. Methods of the invention include contacting the cell, or a nearby cell with an anti-CD44 antibody as described herein, in an amount sufficient to ablate or kill, the cell.

In another aspect, the invention features a method for treating a CD44-expressing cancer, e.g., an HCELL expressing cancer, in a subject. The method includes, for example: administering to a subject an anti-CD44 antibody or antigen binding portion thereof (e.g., an antibody or antigen binding portion thereof as described herein) to thereby treat the cancer. In one embodiment, the anti-CD44 antibody or antigen binding portion thereof inhibits interaction of CD44 with a CD44 ligand. In one embodiment, the antibody further includes a label. In one embodiment, the antibody includes a toxin. In one embodiment, the antibody can mediate complement mediated cytotoxicity or antigen-dependent cellular cytotoxicity. In one embodiment, the method includes administering to the subject an additional agent. For example, the method can include administering a second antibody or antigen binding portion thereof, a chemotherapeutic agent, or a cytotoxic agent. In one embodiment, the cancer is a melanoma, a cancer of the breast, ovaries, colon, or brain.

In another aspect, the invention features a method for increasing an immune response to a cancer in a subject. The method includes, for example: administering to a subject an anti-CD44 antibody or antigen binding portion thereof (e.g., an antibody or antigen binding portion thereof as described herein), to thereby increase the immune response to the cancer. In one embodiment, the antibody or antigen binding portion thereof agonizes interaction of CD44 with a CD44 ligand (e.g., hyaluronan). In one embodiment, the method includes administering to the subject an additional agent. For example, the method can include administering a second antibody or antigen binding portion thereof, a chemotherapeutic agent, a cytotoxic agent, or an immune-modulatory agent (e.g., a recombinant cytokine). In one embodiment, the cancer is a melanoma, a cancer of the breast, ovaries, colon, or brain.

In another aspect, the invention features a method for treating a hematopoietic cancer in a subject. The method includes, for example: administering to a subject an anti-HCELL antibody or antigen binding portion thereof (e.g., an antibody or antigen binding portion thereof as described herein), to thereby treat the cancer. In one embodiment, the cancer is an HCELL-expressing cancer (e.g., an HCELL⁺ lymphoma or and HCELL⁺ leukemia). In one embodiment, the antibody inhibits interaction of HCELL with an HCELL ligand (e.g., L-selectin or E-selectin). In one embodiment, the antibody is coupled to a label, e.g., a radioisotope, or a cytotoxic moiety (e.g., a toxin). In one embodiment, the method includes administering to the subject an additional agent. For example, the method can include administering a second antibody or antigen binding portion thereof, a chemotherapeutic agent, or a cytotoxic agent.

In yet another aspect, the invention features a method for treating an inflammatory or vascular or autoimmune disorder. The method includes, for example: administering to a subject an anti-CD44 antibody or antigen binding portion thereof (e.g., an antibody or antigen binding portion thereof as described herein), to thereby treat the disorder. In one embodiment, the antibody antagonizes interaction of CD44 with a CD44 ligand. In one embodiment, the antibody or antigen binding portion thereof antagonizes one or more of: CD44 interactions with hyaluronan; HCELL interactions with L-selectin; and HCELL interactions with E-selectin. In one embodiment, the disorder is a cutaneous inflammatory or autoimmune disorder (e.g., acute cutaneous graft versus host disease, psoriasis, atopic dermatitis, or contact dermatitis). In one embodiment, the method includes administering to the subject an additional agent. For example, the method can include administering a second antibody or antigen binding portion thereof, a cytotoxic agent, or an anti-inflammatory agent (e.g., a non-steroidal anti-inflammatory drug or a glucocorticosteroid), or an immunosuppressive agent (e.g., cyclosporine).

In another aspect, the invention features a method for treating an inflammatory or autoimmune disorder. The method includes, for example: administering to a subject an anti-HCELL antibody or antigen binding portion thereof (e.g., an antibody or antigen binding portion thereof as described herein), to thereby treat the disorder. In one embodiment, the antibody or antigen binding portion thereof inhibits interaction of HCELL with an HCELL ligand. In one embodiment, the method includes administering to the subject an additional agent. For example, the method can include administering a second antibody or antigen binding portion thereof, a cytotoxic agent, or an anti-inflammatory agent (e.g., a non-steroidal anti-inflammatory drug or a glucocorticosteroid), or an immunosuppressive agent (e.g., cyclosporine). In one embodiment, the disorder is arthritis. In one embodiment, the disorder is graft-versus-host disease.

In another aspect, the invention features a method for treating an infectious disorder. The method includes, for example: administering to a subject an anti-CD44 antibody or antigen binding portion thereof (e.g., an antibody or antigen binding portion thereof as described herein), to thereby treat the disorder. In one embodiment, the antibody or antigen binding portion thereof agonizes interaction of CD44 with a CD44 ligand. In another embodiment, it antagonizes the interaction of CD44 with its ligand(s). In one embodiment, the method includes administering to the subject an additional agent. For example, the method can include administering a second antibody or antigen binding portion thereof, a cytotoxic agent, an anti-inflammatory agent (e.g., a non-steroidal anti-inflammatory drug or a glucocorticosteroid), or an immune-modulatory agent.

In one embodiment, the infectious disorder is infection with an influenza virus. Other viral infections that can be treated include infections with one of the following viruses: human immunodeficiency virus, hepatitis B virus, hepatitis C virus, hepatitis A virus, smallpox, human papilloma viruses, adenoviruses, rhinoviruses, coronaviruses, herpes simplex virus, respiratory syncytial viruses, rabies, and coxsackie virus.

In another aspect, the invention features a method for treating an infectious disorder. The method includes, for example: administering to a subject an anti-HCELL antibody, or antigen binding portion thereof (e.g., an antibody or antigen binding portion thereof as described herein), to thereby treat the disorder. In one embodiment, the antibody or antigen binding portion thereof agonizes interaction of HCELL with an HCELL ligand. In one embodiment, the method includes administering to the subject an additional agent. For example, the method can include administering a second antibody or antigen binding portion thereof, a cytotoxic agent, an anti-inflammatory agent (e.g., a non-steroidal anti-inflammatory drug or a glucocorticosteroid), or an immune-modulatory agent.

In another aspect, the invention features a method for modulating contraception in a subject. The method includes, for example: administering to the subject an anti-HCELL antibody or antigen binding portion thereof (e.g., an antibody or antigen binding portion thereof as described herein), to thereby modulate contraception in the subject. In one embodiment, the antibody or antigen binding portion thereof inhibits HCELL-selectin interactions (e.g., to prevent pregnancy, and/or to reduce the likelihood of implantation). In one embodiment, the antibody or antigen binding portion thereof agonizes HCELL-selectin interactions (e.g., to stimulate conception, and/or to increase the likelihood of implantation). In one embodiment, the method includes administering to the subject a second agent (e.g., a hormone).

In another aspect, the invention features a method for modulating implantation of an embryo in a mammalian subject. The method includes, for example: administering to the subject an anti-HCELL antibody or antigen binding portion thereof (e.g., an antibody or antigen binding portion thereof as described herein), to thereby modulate implantation of an embryo in a subject. In one embodiment, the anti-HCELL antibody or antigen binding portion thereof inhibits HCELL-L-selectin interactions (e.g., to prevent implantation of the embryo and thereby prevent pregnancy). The antibody or antigen binding portion thereof can be administered with one or more additional agents, e.g., agents that decrease the likelihood of implantation.

In another aspect, the invention features a kit including: an antibody or antigen binding portion thereof that modulates an HCELL-selectin interaction (e.g., an antibody or antigen binding portion thereof as described herein); and instructions for administering the antibody to a subject at risk for a disorder characterized by HCELL-selectin interactions. In some embodiments, the kit can include a pharmaceutical composition comprising an antibody and a pharmaceutically acceptable carrier.

In another aspect, the invention features a kit including: an antibody or antigen binding portion thereof that specifically binds to an HCELL glycoprotein (e.g., an antibody or antigen binding portion thereof as described herein), wherein the antibody or antigen binding portion thereof includes at least one of the following characteristics: (a) the antibody or antigen binding portion thereof does not specifically bind to a non-HCELL glycoform of CD44; (b) the antibody or antigen binding portion thereof modulates binding of HCELL to hyaluronan; (c) the antibody or antigen binding portion thereof modulates binding of HCELL to E-selectin; (d) the antibody or antigen binding portion thereof modulates binding of HCELL to L-selectin. The kit can further include instructions, e.g., instructions for use in, e.g., a method of treating disorder described herein

In another aspect, the invention features a kit including: an antibody or antigen binding portion thereof that specifically binds to CD44 (e.g., an antibody or antigen binding portion thereof as described herein); and instructions for administering the antibody to a subject at risk for a disorder characterized by CD44-hyaluronan interactions. In some embodiments, the kit can include a pharmaceutical composition comprising an antibody and a pharmaceutically acceptable carrier.

In another aspect, the invention features a kit including: an antibody or antigen binding portion thereof that specifically binds to CD44 (e.g., an antibody or antigen binding portion thereof as described herein), wherein the antibody or antigen binding portion thereof modulates binding of CD44 to hyaluronan (e.g., increases or decreases CD44 binding to hyaluronan. In one embodiment, the antibody or antigen binding portion thereof modulates binding of HCELL to E-selectin. In one embodiment, the antibody or antigen binding portion thereof modulates binding of HCELL to L-selectin. The kit can further include instructions, e.g., instructions for use in, e.g., a method of treating disorder described herein

In another aspect, the invention provides, compositions, e.g., pharmaceutical compositions, which include a pharmaceutically acceptable carrier, excipient or stabilizer, and at least one of the anti-CD44 antibodies (or portions thereof) described herein. In a preferred embodiment the antibody is conjugated to a label or a therapeutic agent. In one embodiment, the compositions, e.g., the pharmaceutical compositions, comprise a combination of two or more of the aforesaid anti-CD44 antibodies. Combinations of the anti-CD44 antibody and a drug, e.g., a therapeutic agent are also within the scope of the invention.

The invention also features nucleic acid sequences that encode a heavy and light chain of an antibody described herein. For example, the invention features, a first and second nucleic acid encoding a heavy and light chain variable region, respectively, of an anti-CD44 antibody molecule as described herein. In another aspect, the invention features host cells and vectors containing the nucleic acids of the invention.

In another aspect, the invention features a method of producing an anti-CD44 antibody, or antigen binding portion thereof. The method includes: providing a first nucleic acid encoding a heavy chain variable region; providing a second nucleic acid encoding a light chain variable region; and introducing said first and second nucleic acids into a host cell under conditions that allow expression and assembly of said light and heavy chain variable regions.

The first and second nucleic acids can be linked or unlinked, e.g., expressed on the same or different vector, respectively. The host cell can be a eukaryotic cell, e.g., a mammalian cell, an insect cell, a yeast cell, or a prokaryotic cell, e.g., E. coli. For example, the mammalian cell can be a cultured cell or a cell line. Exemplary mammalian cells include lymphocytic cell lines (e.g., NS0), Chinese hamster ovary cells (CHO), COS cells, oocyte cells, and cells from a transgenic animal, e.g., mammary epithelial cell. For example, nucleic acids encoding an antibody described herein can be expressed in a transgenic animal. In one embodiment, the nucleic acids are placed under the control of a tissue-specific promoter (e.g., a mammary specific promoter) and the antibody is produced in the transgenic animal. For example, the antibody molecule is secreted into the milk of the transgenic animal, such as a transgenic cow, pig, horse, sheep, goat or rodent.

The methods of the invention, e.g., methods of treatment or preventing, can further include the step of monitoring the subject, e.g., for a change (e.g., an increase or decrease) in a symptom or marker of the disorder, e.g., tumor size; levels of a cancer marker, the rate of appearance of new lesions, the appearance of new disease-related symptoms; the size of soft tissue mass, e.g., a decreased or stabilization; quality of life, e.g., amount of disease associated pain, e.g., bone pain; or any other parameter related to clinical outcome. The subject can be monitored in one or more of the following periods: prior to beginning of treatment; during the treatment; or after one or more elements of the treatment have been administered. Monitoring can be used to evaluate the need for further treatment with the same anti-CD44 antibody or portion thereof or for additional treatment with additional agents. Generally, a decrease in one or more of the parameters described above is indicative of the improved condition of the subject, although with serum hemoglobin levels, an increase can be associated with the improved condition of the subject.

The anti-CD44 antibody or portion thereof can be used alone in unconjugated form to thereby ablate or kill the CD44-expressing cells by, e.g., antibody-dependent cell killing mechanisms such as complement-mediated cell lysis and/or effector cell-mediated cell killing. In other embodiments, the anti-CD44 antibody or portion thereof can be bound to a substance (such as an antibody-conjugate), e.g., a cytotoxic agent or moiety, e.g., a therapeutic drug, a compound emitting radiation, molecules of plant, fungal, or bacterial origin, or a biological protein (e.g., a protein toxin) or particle (e.g., a recombinant viral particle, e.g., via a viral coat protein). For example, the anti-CD44 antibody, or antigen binding portion thereof, can be coupled to a radioactive isotope such as an α-, β-, or γ-emitter, or a β- and γ-emitter. Examples of radioactive isotopes include iodine (¹³¹I or ¹²⁵I), yttrium (⁹⁰Y), lutetium (¹⁷⁷Lu), actinium (²²⁵Ac), praseodymium, or bismuth (²¹²Bi or ²¹³Bi). Alternatively, the anti-CD44 antibody, or antigen binding portion thereof, can be coupled to a biological protein, a molecule of plant or bacterial origin (or derivative thereof).

The methods and compositions of the invention can be used in combination with other therapeutic modalities. In one embodiment, the methods of the invention include administering to the subject an anti-CD44 antibody or portion thereof as described herein, in combination with a cytotoxic agent, in an amount effective to treat or prevent said disorder. The antibody and the cytotoxic agent can be administered simultaneously or sequentially. In other embodiments, the methods and compositions of the invention are used in combination with surgical and/or radiation procedures. In yet other embodiments, the methods can be used in combination with immunodulatory agents, e.g., IL-1, 2, 4, 6, or 12, or interferon alpha or gamma, or immune cell growth factors such as GM-CSF. In other embodiments, the methods are used in combination with anti-inflammatory or immunosuppressive treatments. Anti-inflammatory agents include non-steroidal anti-inflammatory drugs (e.g., aspirin, ibuprofen, naproxen, and the like), COX-2 inhibitors (e.g., rofecoxib, and celecoxib); leukotriene synthesis inhibitors, leukotriene receptor antagonists, glucocorticosteroids, steroid chemical derivatives, proinflammatory cytokine antagonists, and antibodies to molecules involved in the regulation of immune responses (e.g., anti-CD 3 antibodies, anti-CD4 antibodies). Immunosuppressive agents include cyclosporine, FK-506, dexamethasone, and azathioprine.

In another aspect, the invention features methods for detecting the presence of a CD44 protein in a sample in vitro (e.g., a biological sample, e.g., serum, semen or urine, or a tissue biopsy, e.g., bone marrow). The subject method can be used to evaluate, e.g., diagnose or stage a disorder described herein. The method includes: (i) contacting the sample (and optionally, a reference, e.g., a control sample) with an anti-CD44 antibody, or portion thereof, as described herein, under conditions that allow interaction of the anti-CD44 antibody and the CD44 protein to occur; and (ii) detecting formation of a complex between the anti-CD44 antibody, and the sample (and optionally, the reference, e.g., control, sample). Formation of the complex is indicative of the presence of CD44 protein, and can indicate the suitability or need for a treatment described herein. For example, a statistically significant change in the formation of the complex in the sample relative to the reference sample, e.g., the control sample, is indicative of the presence of CD44 in the sample.

In yet another aspect, the invention provides a method for detecting the presence of CD44 in vivo (e.g., in vivo imaging in a subject). The method can be used to evaluate, e.g., diagnose or stage a disorder described herein, in a subject, e.g., a mammal, e.g., a primate, e.g., a human. The method includes: (i) administering to a subject an anti-CD44 antibody (or antigen binding portion thereof), under conditions that allow interaction of the anti-CD44 antibody (or portion thereof) and the CD44 protein to occur; and (ii) detecting formation of a complex between the antibody or portion and CD44. A statistically significant change in the formation of the complex in the subject relative to the reference, e.g., the control subject or subject's baseline, is indicative of the presence of the CD44.

In other embodiments, a method of diagnosing or staging a disorder as described herein is provided. The method includes: (i) identifying a subject having, or at risk of having, the disorder; (ii) obtaining a sample of a tissue or cell affected with the disorder; (iii) contacting said sample or a control sample with an anti-CD44 antibody as described herein under conditions that allow interaction of the binding agent and the CD44 protein to occur, and (iv) detecting formation of a complex. A statistically significant increase in the formation of the complex between the antibody (or portion thereof) with respect to a reference sample, e.g., a control sample, is indicative of the disorder or the stage of the disorder.

Preferably, the anti-CD44 antibody or portion thereof, used in the in vivo and in vitro diagnostic methods is directly or indirectly labeled with a detectable substance to facilitate detection of the bound or unbound binding agent. Suitable detectable substances include various biologically active enzymes, prosthetic groups, fluorescent materials, luminescent materials, paramagnetic (e.g., nuclear magnetic resonance active) materials, and radioactive materials. In some embodiments, the anti-CD44 antibody or portion thereof is coupled to a radioactive ion, e.g., indium (¹¹¹In), iodine (¹³¹I or ¹²⁵I), yttrium (⁹⁰Y), lutetium (¹⁷⁷Lu), actinium (²²⁵Ac), bismuth (²¹²Bi or ²¹³Bi), sulfur (³⁵S), carbon (¹⁴C), tritium (³H), rhodium (¹⁸⁸Rh), technetium (⁹⁹mTc), praseodymium, or phosphorous (³²p).

Other features and advantages of the instant invention will become more apparent from the following detailed description and claims.

### DETAILED DESCRIPTION OF THE INVENTION

This invention provides, inter alia, antibodies, or antigen binding portions thereof, to CD44 or specific glycoforms of CD44, e.g., an HCELL glycoform of CD44. The anti-CD44 antibodies can modulate the binding of CD44, e.g., specific glycoforms of CD44 (e.g., HCELL), to its ligand, e.g., L-selectin, E-selectin, or hyaluronan. Anti-CD44 (e.g., anti-HCELL antibodies) which modulate one or more of (a) binding to hyaluronan, (b) binding to L-selectin, and (c) binding to E-selectin can be used for a variety of applications in vitro and in vivo. In addition, this invention provides for antibodies that recognize glycosylations such as fucose and/or sialic acid on CD44 which define subpopulations of cells bearing such CD44 glycoforms. For example, antibodies that specifically bind HCELL can be used to isolate HCELL-expressing hematopoietic progenitor cells (HPC) from a population of HPC. Enrichment of HPC in this manner can produce a population of HPC with higher engraftment potential than a non-enriched population. Anti-HCELL antibodies that agonize, or increase the ability of an HCELL to bind an HCELL ligand (e.g., L-selectin or E-selectin) can also increase the engraftment potential of HPC. In another embodiment, anti-CD44, e.g., anti-HCELL antibodies or antigen biding portions thereof can be used to enhance homing of stem cells to damaged tissues for regenerative therapies. In another embodiment, the anti-CD44, e.g., anti-HCELL antibodies can be used to diagnose, evaluate, and/or treat CD44 (e.g., HCELL)-expressing cancers, e.g., HCELL-expressing leukemias, lymphomas, and myelodysplastic syndromes. Anti-CD44, e.g., anti-HCELL antibodies can be used in other L-selectin and E-selectin dependent processes. For example, the antibodies can be used to inhibit or enhance L-selectin interactions that occur during contraception and implantation of the embryo after fertilization.

Anti-CD44 antibodies that inhibit interaction of CD44 with a CD44 ligand can be used to block the activity of CD44-expressing cells by blocking contact of the CD44-expressing cells with their targets, e.g., to treat acute and chronic inflammatory and autoimmune diseases (e.g., psoriasis, atopic dermatitis, or arthritis). Anti-CD44 antibodies that agonize CD44-CD44 ligand interactions can be used to augment immune responses, e.g., to treat infectious diseases.

In order that the present invention may be more readily understood, certain terms are first defined. Additional definitions are set forth throughout the detailed description. The term “CD44” as used herein refers to mammalian CD44, preferably human CD44. CD44 (also known as HCAM, Pgp-1 and Hermes antigen) is a polymorphic integral cell membrane glycoprotein expressed on lymphocytes, hematopoietic progenitor cells, and nonhematopoietic cells. Human CD44 includes several protein products, including CD44S, CD44H, HCELL, CD44R1, CD44R2, which are encoded by alternating spliced mRNA variants of the CD44 nucleic acid disclosed in Screaton, G. R., et al., Proc. Natl. Acad. Sci. U.S.A. 89 (24), 12160-12164 (1992) (genomic sequence). The term also refers to the various glycoforms of CD44. The term also refers to an amino acid sequence encoded by a nucleic acid sequence that hybridizes to a nucleic acid sequence disclosed in Screaton, G. R., et al. (Proc. Natl. Acad. Sci. U.S.A. 89 (24), 12160-12164 (1992)) under stringent conditions, e.g., highly stringent conditions.

The gene encoding human CD44 consists of 20 exons (19 exons in earlier literature, exons 6a and 6b have been reclassified as exons 6 and 7, to make 20 exons total). Although a single gene located on the short arm of human chromosome 11 encodes CD44, multiple mRNA transcripts that arise from the alternative splicing of 12 of the 20 exons have been identified. The standard and most prevalent form of CD44 (termed CD44s) consists of a protein encoded by exons 1-5,16-18, and 20. This form is the most predominant form on hematopoietic cells, and is also known as CD44H. CD44s exhibits the extracellular domains (exons 1-5 and 16), the highly conserved transmembrane domain (exon 18), and the cytoplasmic domain (exon 20). The 1482 bp of open reading frame mRNA for human CD44s results in translation of a polypeptide chain of ˜37 kDa. Post-translational addition of N-linked and O-linked oligosaccharides contribute to the ˜85-kDa molecular mass of the final CD44 protein as estimated by SDS-PAGE.

The standard or hematopoietic isoform of CD44 (CD44H) is a type 1 transmembrane molecule consisting of ˜270 amino acids (aa) of extracellular domain (including 20 aa of leader sequence, a 21 aa transmembrane domain and a 72 aa cytoplasmic domain). The amino terminal ˜180 aa are conserved among mammalian species (˜85% homology). This region contains six conserved cysteines, and six conserved consensus sites for N glycosylation. Five conserved consensus sites for N-glycosylation are located in the amino terminal 120 aa of CD44. All five sites appear to be utilized in murine and human cell lines. Several studies have shown that cell specific N-glycosylation can modulate the HA binding function of CD44. Cell lines and normal B-cells showed differences in N-glycosylation associated with different HA binding states. In particular, CD44 from HA binding cells had less glycosylation than from non-HA binding cells. Additionally, removal of sialic acids (both from the cell surface and from CD44-Ig fusion proteins) enhances HA binding.

In contrast, the non-conserved region (˜aa 183 to 256) shows only ˜35% similarity between mammalian species. This region contains potential sites for numerous carbohydrate modifications of CD44 and the site of alternative splicing which allows for the insertion of extra amino acid sequence from variable exons of the CD44 gene.

A HCELL polypeptide comprises an amino acid sequence of CD44 (in standard form or as a splice variant) and is glycosylated such that it interacts with E-selectin and/or L-selectin. For example, a HCELL polypeptide can be a polypeptide that interacts with an antibody having the binding specificity of monoclonal antibody HECA-452 (ATCC Number: HB-11485). HECA-452 recognizes cutaneous lymphocyte associated antigen. HECA-452 binding of HCELL decreases after N-glycosidase-F, sialidase or fucosidase treatment. Furthermore, HCELL activity, e.g., E-selectin and L-selectin binding also decreases upon N-glycosidase-F, sialidase, or fucosidase treatment demonstrating the importance of the sialofucosylated N-linked glycans in HCELL function. In contrast, sialylation of CD44 inhibits binding of CD44 to hyaluronic acid, but HCELL glycoforms of CD44 retain hyaluronic acid binding. Moreover, CD44 binding to hyaluronan is increased by sulfation, but sulfation is not necessary for the E- and L-selectin activity of HCELL.

In one embodiment, the CD44 polypeptide is the standard or hematopoietic isoform of CD44 (CD44H). Alternately, the CD44 polypeptide may be an alternatively spliced isoform such as the R1 (CD44R1) or R2 isoform (CD44R2). In other embodiments, the CD44 can be an HCELL polypeptide, e.g., a polypeptide described in PCT Publication No. WO 02/44342. In other embodiments, the CD44 polypeptide is a known variant or glycoform.

Ligands of CD44 include hyaluronan, E-selectin, L-selectin, fibronectin, and collagen.

An “anti-CD44 antibody” is an antibody that interacts with (e.g., binds to) CD44, preferably human CD44. Preferably, the anti-CD44 antibody interacts with, e.g., binds to, the extracellular domain of CD44, e.g., the extracellular domain of human CD44, e.g., a portion of the protein encoded by one or more of exons 1-17. An anti-CD44 antibody may bind to an epitope, e.g., a conformational or a linear epitope.

In a preferred embodiment, the interaction, e.g., binding, occurs with high affinity (e.g., affinity constant of at least 10⁷ M⁻¹, preferably, between 10⁸ M⁻¹ and 10¹⁰ M⁻¹, or about 10⁹ M⁻¹) and specificity. In some embodiments, the anti-CD44 antibody treats, e.g., ablates or kills a cell, e.g., a CD44-expressing cell (e.g., a hematopoietic progenitor cell or lymphocyte). In one embodiment, the anti-CD44 antibody may bind to and be internalized with the CD44 expressed in the cells and/or vascular endothelial cells proximate to the cells. In those embodiments, the anti-CD44 antibody can be used to target a second moiety, e.g., a labeling agent, to the cell. In other embodiments, the anti-CD44 antibody may mediate host-mediated-killing, e.g., complement- or ADCC-mediated killing, of the cell and/or the cell proximate thereto, upon binding to the extracellular domain of CD44. The cell can be killed directly by the anti-CD44 antibody by binding directly to the cell or the cells proximate thereto.

As used herein, the term “treat” or “treatment” is defined as the application or administration of an anti-CD44 antibody or antigen binding portion thereof to a subject, e.g., a patient, or application or administration to an isolated tissue or cell from a subject, e.g., a patient, which is returned to the patient. The anti-CD44 antibody or antigen binding portion thereof, can be administered alone or in combination with, a second agent. The subject can be a patient having a disorder (e.g., a disorder as described herein), a symptom of a disorder or a predisposition toward a disorder. The treatment can be to cure, heal, alleviate, relieve, alter, remedy, ameliorate, palliate, improve or affect the disorder, the symptoms of the disorder or the predisposition toward the disorder.

As used herein, an amount of an anti-CD44 antibody effective to treat a disorder, or a “therapeutically effective amount” refers to an amount of the antibody which is effective, upon single or multiple dose administration to a subject, in treating a cell, e.g., a hematopoietic cell, a lymphocyte, or cancer cell (e.g., a CD44-expressing cell), or in prolonging curing, alleviating, relieving or improving a subject with a disorder as described herein beyond that expected in the absence of such treatment. Treating can include embodiments in which cells of a subject or a donor are manipulated with antibodies ex vivo, and reintroduced into a subject.

As used herein, an amount of an anti-CD44 antibody effective to prevent a disorder, or a “a prophylactically effective amount” of the antibody refers to an amount of an anti-CD44 antibody, e.g., an anti-CD44 antibody as described herein, which is effective, upon single- or multiple-dose administration to the subject, in preventing or delaying the occurrence of the onset or recurrence of a disorder, or treating a symptom thereof.

The terms “agonize”, “induce”, “inhibit”, “potentiate”, “elevate”, “increase”, “decrease” or the like, e.g., which denote quantitative differences between two states, refer to a difference, e.g., a statistically or clinically significant difference, between the two states.

As used herein, “specific binding” refers to the property of the antibody to: (1) to bind to CD44, e.g., human CD44 protein, and/or a specific glycoform of CD44 with an affinity of at least 1×10⁷ M⁻¹, and (2) preferentially bind to CD44, e.g., human CD44 protein, with an affinity that is at least two-fold, 50-fold, 100-fold, 1000-fold, or more greater than its affinity for binding to a non-specific antigen (e.g., BSA, casein) other than CD44.

As used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). Preferably, each VH and VL is composed of three CDRs and four FRs, arranged from amino-terminus to carboxy-terminus in the following order: FR1, CDR1, FR2, CDR2, FR3, CDR3, FR4.

The VH or VL chain of the antibody can further include all or part of a heavy or light chain constant region. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system. The term “antibody” includes intact immunoglobulins of types IgA, IgG, IgE, IgD, IgM (as well as subtypes thereof), wherein the light chains of the immunoglobulin may be of types kappa or lambda.

As used herein, the term “immunoglobulin” refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 Kd or 214 amino acids) are encoded by a variable region gene at the NH2-terminus (about 110 amino acids) and a kappa or lambda constant region gene at the COOH-terminus. Full-length immunoglobulin “heavy chains” (about 50 Kd or 446 amino acids), are similarly encoded by a variable region gene (about 116 amino acids) and one of the other aforementioned constant region genes, e.g., gamma (encoding about 330 amino acids). The term “immunoglobulin” includes an immunoglobulin having: CDRs from a non-human source, e.g., from a non-human antibody, e.g., from a mouse immunoglobulin or another non-human immunoglobulin, from a consensus sequence, or from a sequence generated by phage display, or any other method of generating diversity; and having a framework that is less antigenic in a human than a non-human framework, e.g., in the case of CDRs from a non-human immunoglobulin, less antigenic than the non-human framework from which the non-human CDRs were taken. The framework of the immunoglobulin can be human, humanized non-human, e.g., a mouse, framework modified to decrease antigenicity in humans, or a synthetic framework, e.g., a consensus sequence.

As used herein, “isotype” refers to the antibody class (e.g., IgM or IgG1) that is encoded by heavy chain constant region genes.

The term “antigen binding portion” of an antibody (or simply “antibody portion,” or “portion”), as used herein, refers to a portion of an antibody which specifically binds to CD44 (e.g., human CD44), e.g., a molecule in which one or more immunoglobulin chains is not full length but which specifically binds to CD44 (e.g., human CD44). Examples of binding portions encompassed within the term “antigen binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR) having sufficient framework to specifically bind, e.g., an antigen binding portion of a variable region. An antigen binding portion of a light chain variable region and an antigen binding portion of a heavy chain variable region, e.g., the two domains of the Fv fragment, VL and VH, can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen binding portion” of an antibody. These antibody portions are obtained using conventional techniques known to those with skill in the art, and the portions are screened for utility in the same manner as are intact antibodies.

The term “monospecific antibody” refers to an antibody that displays a single binding specificity and affinity for a particular target, e.g., epitope. This term includes a “monoclonal antibody” or “monoclonal antibody composition,” which as used herein refer to a preparation of antibodies or portions thereof of single molecular composition.

The term “recombinant” antibody, as used herein, refers to antibodies that are prepared, expressed, created or isolated by recombinant means, such as antibodies expressed using a recombinant expression vector transfected into a host cell, antibodies isolated from a recombinant, combinatorial antibody library, antibodies isolated from an animal (e.g., a mouse) that is transgenic for human immunoglobulin genes or antibodies prepared, expressed, created or isolated by any other means that involves splicing of human immunoglobulin gene sequences to other DNA sequences. Such recombinant antibodies include humanized, CDR grafted, chimeric, in vitro generated (e.g., by phage display) antibodies, and may optionally include constant regions derived from human germline immunoglobulin sequences.

As used herein, the term “substantially identical” (or “substantially homologous”) is used herein to refer to a first amino acid or nucleotide sequence that contains a sufficient number of identical or equivalent (e.g., with a similar side chain, e.g., conserved amino acid substitutions) amino acid residues or nucleotides to a second amino acid or nucleotide sequence such that the first and second amino acid or nucleotide sequences have similar activities. In the case of antibodies, the second antibody has the same specificity and has at least 50% of the affinity of the same.

Calculations of “homology” between two sequences can be performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent homology between two sequences can be accomplished using a mathematical algorithm. In a preferred embodiment, the percent homology between two amino acid sequences is determined using the Needleman and Wunsch (1970), J. Mol. Biol. 48:444-453, algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent homology between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a homology limitation of the invention) are a Blossum 62 scoring matrix with a gap penalty of 12, a gap extend penalty of 4, and a frameshift gap penalty of 5.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

It is understood that the antibodies and antigen binding portion thereof of the invention may have additional conservative or non-essential amino acid substitutions, which do not have a substantial effect on the polypeptide functions. Whether or not a particular substitution will be tolerated, i.e., will not adversely affect desired biological properties, such as binding activity can be determined as described in Bowie, J U et al. (1990) Science 247:1306-1310. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

A “non-essential” amino acid residue is a residue that can be altered from the wild-type sequence of the binding agent, e.g., the antibody, without abolishing or more preferably, without substantially altering a biological activity, whereas an “essential” amino acid residue results in such a change.

Anti-CD44 Antibodies

Many types of anti-CD44 antibodies, or antigen binding portions thereof, are useful in the methods of this invention. In one embodiment, the anti-CD44 antibody specifically recognizes an HCELL glycoform of CD44. The antibody can also have one or more of the following characteristics: (a) the antibody or antigen binding portion thereof does not specifically bind to an -HCELL glycoform of CD44; (b) the antibody or antigen binding portion thereof modulates binding of HCELL to hyaluronate; (c) the antibody or antigen binding portion thereof modulates binding of HCELL to E-selectin; (d) the antibody or antigen binding portion thereof modulates binding of HCELL to L-selectin. “Modulating” can refer to agonizing or inhibiting binding.

The antibodies can be of the various isotypes, including: IgG (e.g., IgG1, IgG2, IgG3, IgG4), IgM, IgA1, IgA2, IgD, or IgE. Preferably, the antibody is an IgG isotype, e.g., IgG1. The antibody molecules can be full-length (e.g., an IgG1 or IgG4 antibody) or can include only an antigen binding portion (e.g., a Fab, F(ab′)₂, Fv or a single chain Fv portion). These include monoclonal antibodies, recombinant antibodies, chimeric antibodies, and humanized antibodies, as well as antigen binding portions of the foregoing.

Monoclonal or polyclonal anti-CD44 antibodies can be used in the methods of the invention. Additionally, any reagent (e.g., phagemid) recognizing CD44 can be used in the methods of the invention. Preferably, the antibodies or other reagents bind to the extracellular domain of CD44 (i.e., an epitope of CD44 located outside of a cell).

Additional monoclonal antibodies to CD44 can be generated using techniques known in the art. Monoclonal antibodies can be produced by a variety of techniques, including conventional monoclonal antibody methodology e.g., the standard somatic cell hybridization technique of Kohler and Milstein, Nature 256: 495 (1975). See generally, Harlow, E. and Lane, D. (1988) Antibodies: A Laboratory Manual, Cold Spring Harbor Laboratory Press, Cold Spring Harbor, N.Y.

Useful immunogens for the purpose of this invention include CD44 (e.g., human CD44-bearing cells (e.g., KG1a cells, lymphocytes, CD34⁺ hematopoietic cells); membrane fractions of CD44 -expressing cells; isolated or purified CD44, e.g., human CD44 protein (e.g., biochemically isolated CD44, or a portion thereof, e.g., the extracellular domain of CD44, or secreted forms of CD44 produced by linking of a portion of CD44 with immunoglobulin products such as CD44-Ig chimeras).

In one embodiment, anti-CD44 (e.g., anti-HCELL) antibodies are identified using a phage display library. A typical antibody display library displays a polypeptide that includes a VH domain and a VL domain. Antibody libraries can be constructed by a number of processes (see, e.g., de Haard et al. (1999) J. Biol. Chem 274:18218-30; Hoogenboom et al. (1998) Immunotechnology 4:1-20. and Hoogenboom et al. (2000) Immunol Today 21:371-8.

In display phage, the polypeptide component (i.e., the antibody) is typically fused to a coat protein of bacteriophage resulting from translation of a nucleic acid encoding the polypeptide component fused to the coat protein. The linkage can include a flexible peptide linker, a protease site, or an amino acid incorporated as a result of suppression of a stop codon. Phage display is described, for example, in Ladner et al., U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; WO 90/02809; de Haard et al. (1999) J. Biol. Chem 274:18218-30; Hoogenboom et al. (1998) Immunotechnology 4:1-20; Hoogenboom et al. (2000) Immunol Today 2:371-8; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Rebar et al. (1996) Methods Enzymol. 267:129-49; Hoogenboom et al. (1991) Nuc Acid Res 19:4133-4137; and Barbas et al. (1991) PNAS 88:7978-7982.

Phage display systems have been developed for filamentous phage (phage fl, fd, and M13) as well as other bacteriophage (e.g. T7 bacteriophage and lambdoid phages; see, e.g., Santini (1998) J. Mol. Biol. 282:125-135; Rosenberg et al. (1996) Innovations 6:1-6; Houshmet al. (1999) Anal Biochem 268:363-370). The filamentous phage display systems typically use fusions to a minor coat protein, such as gene III protein, and gene VIII protein, a major coat protein, but fusions to other coat proteins such as gene VI protein, gene VII protein, gene IX protein, or domains thereof can also been used (see, e.g., WO 00/71694).

A phagemid system can be utilized for identifying an anti-CD44 antibody in an antibody library. In a phagemid system, the nucleic acid encoding the polypeptide component fused to e.g., gene III, provided on a plasmid. The plasmid includes a phage origin of replication so that the plasmid is incorporated into bacteriophage particles when bacterial cells bearing the plasmid are infected with helper phage, e.g. M13K01. The helper phage provides an intact copy of gene III and other phage genes required for phage replication and assembly. The helper phage has a defective origin such that the helper phage genome is not efficiently incorporated into phage particles relative to the plasmid that has a wild type origin.

Anti-CD44 antibodies or portions thereof useful in the present invention may also be recombinant antibodies produced by host cells transformed with DNA encoding immunoglobulin light and heavy chains of a desired antibody. Recombinant antibodies may be produced by known genetic engineering techniques. For example, recombinant antibodies may be produced by cloning a nucleotide sequence, e.g., a cDNA or genomic DNA, encoding the immunoglobulin light and heavy chains of the desired antibody. The nucleotide sequence encoding those polypeptides is then inserted into expression vectors so that both genes are operatively linked to their own transcriptional and translational expression control sequences. The expression vector and expression control sequences are chosen to be compatible with the expression host cell used. Typically, both genes are inserted into the same expression vector. Prokaryotic or eukaryotic host cells may be used.

Expression in eukaryotic host cells is preferred because such cells are more likely than prokaryotic cells to assemble and secrete a properly folded and immunologically active antibody. However, any antibody produced that is inactive due to improper folding may be renaturable according to known methods (Kim and Baldwin, “Specific Intermediates in the Folding Reactions of Small Proteins and the Mechanism of Protein Folding”, Ann. Rev. Biochem. 51, pp. 459-89 (1982)). It is possible that the host cells will produce portions of intact antibodies, such as light chain dimers or heavy chain dimers, which also are antibody homologs according to the present invention.

It will be understood that variations on the above procedure are useful in the present invention. For example, it may be desired to transform a host cell with DNA encoding either the light chain or the heavy chain (but not both) of an antibody. Recombinant DNA technology may also be used to remove some or all of the DNA encoding either or both of the light and heavy chains that is not necessary for CD44 binding, e.g., the constant region may be modified by, for example, deleting specific amino acids. The molecules expressed from such truncated DNA molecules are useful in the methods of this invention. In addition, bifunctional antibodies may be produced in which one heavy and one light chain are anti-CD44 antibody and the other heavy and light chain are specific for an antigen other than CD44, or another epitope of CD44.

Chimeric antibodies can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted (see Robinson et al., International Patent Publication PCTJUS86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

An antibody or an immunoglobulin chain can be humanized by methods known in the art. Once the murine antibodies are obtained, the variable regions can be sequenced. The location of the CDRs and framework residues can be determined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). The light and heavy chain variable regions can, optionally, be ligated to corresponding constant regions.

Murine anti-CD44 antibodies (or anti-CD44 antibodies produced in any other animal) can be sequenced using art-recognized techniques. Humanized or CDR-grafted antibody molecules or immunoglobulins can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Moreover, humanized polyclonal antibodies are also provided herein.

Winter describes a CDR-grafting method that may be used to prepare the humanized antibodies of the present invention (UK Patent Application GB 2188638A, filed on Mar. 26, 1987; Winter U.S. Pat. No. 5,225,539), the contents of which is expressly incorporated by reference. All of the CDRs of a particular human antibody may be replaced with at least a portion of a non-human CDR or only some of the CDRs may be replaced with non-human CDRs. It is only necessary to replace the number of CDRs required for binding of the humanized antibody to a predetermined antigen.

Humanized antibodies can be generated by replacing sequences of the Fv variable region that are not directly involved in antigen binding with equivalent sequences from human Fv variable regions. General methods for generating humanized antibodies are provided by Morrison, S. L., 1985, Science 229:1202-1207, by Oi et al., 1986, BioTechniques 4:214, and by Queen et al. U.S. Pat. No. 5,585,089, U.S. Pat. No. 5,693,761 and U.S. Pat. No. 5,693,762, the contents of all of which are hereby incorporated by reference. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a predetermined target, as described above. The recombinant DNA encoding the humanized antibody, or portion thereof, can then be cloned into an appropriate expression vector.

Also within the scope of the invention are humanized antibodies in which specific amino acids have been substituted, deleted or added. In particular, preferred humanized antibodies have amino acid substitutions in the framework region, such as to improve binding to the antigen. For example, a selected, small number of acceptor framework residues of the humanized immunoglobulin chain can be replaced by the corresponding donor amino acids. Preferred locations of the substitutions include amino acid residues adjacent to the CDR, or which are capable of interacting with a CDR (see e.g., U.S. Pat. No. 5,585,089). Criteria for selecting amino acids from the donor are described in U.S. Pat. No. 5,585,089, e.g., columns 12-16 of U.S. Pat. No. 5,585,089, the contents of which are hereby incorporated by reference. The acceptor framework can be a mature human antibody framework sequence or a consensus sequence. In addition, alterations to the Fc portion or other regions that confer antibody functions such as complement fixation and ADCC are within the scope of the invention.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence. Other techniques for humanizing antibodies are described in Padlan et al. EP 519596 A1, published on Dec. 23, 1992.

The anti-CD44 antibody, or antigen portion thereof, may also be modified by specific deletion of human T cell epitopes or “deimmunization” by the methods disclosed in WO 98/52976 and WO 00/34317, the contents of which are incorporated by reference herein. Briefly, the murine heavy and light chain variable regions of an anti-CD44 antibody can be analyzed for peptides that bind to MSC Class II; these peptides represent potential T-cell epitopes (as defined in WO 98/52976 and WO 00/34317). For detection of potential T-cell epitopes, a computer modeling approach termed “peptide threading” can be applied, and in addition a database of human MHC class II binding peptides can be searched for motifs present in the murine V_{H} and V_{L} sequences, as described in WO 98/52976 and WO 00/34317. These motifs bind to any of the 18 major MHC class II DR allotypes, and thus constitute potential T cell epitopes. Potential T-cell epitopes detected can be eliminated by substituting small numbers of amino acid residues in the variable regions, or preferably, by single amino acid substitutions. As far as possible conservative substitutions are made, often but not exclusively, an amino acid common at this position in human germline antibody sequences may be used. Human germline sequences are disclosed in Tomlinson, I. M. et al. (1992) J. Mol. Biol. 227:776-798; Cook, G. P. et al. (1995) Immunol. Today Vol. 16 (5): 237-242; Chothia, D. et al. (1992) J. Mol. Bio. 227:799-817. The V BASE directory provides a comprehensive directory of human immunoglobulin variable region sequences (compiled by Tomlinson, I. M. et al. MC Centre for Protein Engineering, Cambridge, UK). After the deimmunized V_{H} and V_{L} of an anti-CD44 antibody are constructed by mutagenesis of the murine V_{H} and V_{L} genes. The mutagenized variable sequence can, optionally, be fused to a human constant region, e.g., human IgG1 or κ constant regions.

Antibody Assays

Anti-CD44 antibodies can be assayed for the ability to modulate a CD44 activity, e.g., a binding activity. To determine the activity of an anti-CD44 antibody, assays for CD44 binding or cell binding can be carried out. A screen (e.g., a high throughput screen) for antagonist or agonist antibodies can be carried out by: (a) binding one or more types of substrate proteins (e.g., CD44) or peptides to a solid support (e.g., the wells of microtiter plates); (b) exposing the substrate to a blocking agent (standard blocking agents are known); and (c) exposing the substrate to a CD44 ligand (e.g., hyaluronan, L-selectin, E-selectin) and a test antibody. Alternatively, the CD44 ligand can be bound to a solid support, and CD44 can be added and tested for binding to hyaluronan, L-selectin, or E-selectin).

The components of the reaction are typically supplied in a buffered solution and the reaction is allowed to proceed at a temperature (the temperature can vary from, for example, 4° C. to room temperature (about 23° C.) to a physiological temperature (about 37° C.)) and for a period of time that is in the linear range of the assay. The reaction can be terminated in a number of ways (by, for example, rinsing the support several times with a buffered solution), and binding can be determined (standard techniques are available to measure, for example, radioactive tags). Antagonist antibodies are identified as the agents that reduce the extent to which the CD44 ligand was able to bind the CD44 as compared to the extent to which the CD44 ligand binds CD44 in the absence of the antibody. Agonists increase the extent to which the CD44 ligand binds the CD44 as compared to the extent to which the CD44 ligand binds CD44 in the absence of the antibody.

Appropriate controls can be carried out in connection with any of the methods of the invention. For example, the method described above can be carried out in the presence and absence of a test antibody (representing experimental and control paradigms, respectively). Alternatively, test antibodies and controls (e.g., biologically inactive antibodies, such as denatured or non-specific antibodies) can be used.

CD44-ligand interactions can also be assayed in cell-based systems. These methods can be carried out by, for example, contacting a cell that expresses a CD44 protein with a test antibody and assessing the ability of the test antibody to inhibit binding to a ligand either as an isolated molecule or expressed on another cell. The cell can be any biological cell that expresses or binds (e.g., an presents) the CD44 and/or the CD44 ligand whether naturally or as a result of genetic engineering. For example, the cell can be a mammalian cell, such as a murine, canine, ovine, porcine, or human cell. The cell can also be non-mammalian (e.g., a Drosophila cell).

As discussed above, the assays performed can reveal whether an anti-CD44 antibody inhibits or increases the ability of CD44 to simply bind to, or otherwise associate with, another molecule or moiety, e.g., a CD44 ligand. For example, one can determine whether an antibody inhibits or increases the ability of hyaluronan to bind to a substrate or a component of a cell. These methods can be carried out by, for example, labeling either the CD44 or the CD44 ligand with a marker, such as a radioisotope or enzymatic label, so that ligand-containing moieties can be detected. Suitable labels are known in the art and include, for example, ¹²⁵I, ³⁵S, ¹⁴C, or ³H (which are detectable by direct counting of radioemmissions or by scintillation counting). Enzymatic labels include horseradish peroxidase, alkaline phosphatase, and luciferase, which are detected by determining whether an appropriate substrate of the labeling enzyme has been converted to product. Fluorescent labels can also be used. Another way to detect interaction (between any two molecules) using a fluorophore is by fluorescence (resonance) energy transfer (FET or FRET) (see, e.g., Lakowicz et al., U.S. Pat. No. 5,631,169 and Stavrianopoulos et al., U.S. Pat. No. 4,868,103). A fluorophore label on the first, or “donor,” molecule emits fluorescent energy that is absorbed by a fluorescent label on the second, or “acceptor,” molecule, which fluoresces due to the absorbed energy (the labels on the two molecules emitting different, and therefore distinguishable, wavelengths of light). Alternately, the “donor” protein can simply utilize the natural fluorescent energy of tryptophan residues. Since the efficiency of energy transfer between the labels is related to the distance separating them, the spatial relationship between the molecules can be assessed. Where the two molecules bind one another, emission from the acceptor molecule is maximal; emission can be measured readily (with, for example, a fluorimeter).

As noted above, the activity of the antibodies can be evaluated in assays where a CD44 or CD44 ligand is bound to a solid support. More generally, wherever CD44-related binding is assessed, one of the binding partners can be anchored to a solid phase (e.g., a microtiter plate, a test tube (e.g., a microcentrifuge tube) or a column). The non-anchored binding partner can be labeled, either directly or indirectly, with a detectable label (including any of those discussed herein), and binding can be assessed by detecting the label. If desired, the CD44 can be linked to a moiety (e.g., protein) that binds a matrix. For example, one can identify an anti-CD44 antibody with antagonist activity by crosslinking hyaluronan to a protein, or by fusing a CD44 ligand to glutathione-S-transferase; absorbing the fusion protein to a support (e.g., glutathione sepharose beads (Sigma Chemical, St. Louis, Mo.) or glutathione-derivatized microtiter plates); exposing the immobilized fusion protein to a potential binding partner (e.g., a potential anti-CD44 antagonist antibody) washing away unbound material; and detecting bound material. The exposure should take place under conditions conducive to complex formation (e.g., a physiologically acceptable condition). Alternatively, the complexes can be dissociated from the matrix, and the level of hyaluronan binding can be determined using standard techniques.

CD44 or molecules with which they interact (e.g., CD44 substrates) or which with they may interact (e.g., potential antagonists) can also be immobilized on matrices using biotin and avidin or streptavidin. For example, biotinylated CD44 or molecules to which they bind can be prepared from biotin-NHS (N-hydroxy-succinimide) using techniques known in the art (e.g., using the biotinylation kit sold by Pierce Chemicals, Rockford, Ill.), and immobilized in the wells of avidin- or streptavidin-coated 96 well plates (Pierce Chemical). Regardless of the precise way in which CD44 or a CD44 ligand is immobilized, the CD44 is exposed to a potential binding partner, any unreacted components are removed (e.g., by washing; under conditions that retain any complexes); and the remaining complexes are detected (by virtue of a label or with an antibody (e.g., an antibody that specifically binds CD44, is used in the assay). Although somewhat more labor intensive, the step of detecting CD44 (or a CD44-containing protein complex) can also be carried out by enzyme-linked assays, which rely on detecting an enzymatic activity associated with the CD44 or its target molecule.

Where the binding assay is carried out in a liquid phase, the reaction products (e.g., hyaluronan-containing complexes) can be separated from unreactive components by, for example: differential centrifugation (see, e.g., Rivas and Minton, Trends Biochem. Sci. 18:284-287, 1997); chromatography (gel filtration chromatography, ion-exchange chromatography); electrophoresis (see, e.g., Ausubel et al., Eds. Current Protocols in Molecular Biology 1999, J. Wiley & Sons, New York.); and immunoprecipitation (as described, for example, in Ausubel, supra). Where FET is utilized (see above), further purification is not required. Assays that measure cell adherence under conditions of shear stress can be used to identify agents that agonize or inhibit physiologic CD44, e.g., HCELL, interactions with a ligand. An assay that measures cell adherence can be performed. A cell that expresses CD44, e.g., HCELL can be identified by providing a ligand of CD44, e.g., HCELL (e.g., hyaluronan, L-selectin, E-selectin) immobilized on a solid phase e.g., glass, plastic or membrane, and contacting the solid phase with a fluid sample containing a suspension of test cells. Alternatively, a CD44-ligand-expressing cell can be provided, and contacted to a solid phase containing immobilized CD44. In some aspects the fluid sample is moving. By a moving fluid sample under shear stress, it is meant that the sample flows across the surface of the membrane. Interactions between fluid sample in flow and immobilized ligand can be examined under a wide range of defined flow conditions, ranging from static incubation through physiological levels of shear flow, static conditions and serial application of static and shear conditions, and into supraphysiologic shear levels. For example, shear flow conditions is a shear stress greater than 0.1 dynes/cm². Alternatively, shear flow condition is a shear stress at least 2.8 dynes/cm². Additionally, shear flow condition is a shear stress of at least 9.0 dynes/cm². In some aspects, the fluid moves across the membrane such that physiological shear stress is achieved at the surface. The interaction between the solid phase and the cells is then determined. Such assays can be conducted to compare adherence in the presence of anti-CD44 antibodies (e.g., having agonist or inhibitory activity to a binding interaction of a CD44). Selectin binding activity is preferably measured under shear conditions, as described above.

The interaction between the cells and the solid phase can be, e.g., rolling, firm attachments or specific interaction. In some aspects, the specific interaction is determined by the affinity coefficient. For example a specific interaction is an interaction that has a K_{d} is in the range of 0.1 mM to 7 mM. Preferably, the K_{d} is greater than 1 mM. A cell/agent interaction or alternately a cellsolid phase interaction can be determined for example, by visual inspection under a microscope, colormetrically, flourometrically, by flow cytometry or using a parallel plate flow chamber assay. Alternatively, the interaction is analyzed by labeling the cells, CD44, CD44-ligand, or the antibody using florescent labels, biotin, enzymes such as alkaline phosphatase, horseradish peroxidase or beta-galactosidase, radioactive isotopes or other labels known in the art. The label can be added to the cells, CD44 polypeptide, the CD44 ligand, or the antibody prior or subsequent to contacting the test cell population with the antibody. The membrane or solid phase can then be subject to visualization by microscopy, or by spectrophotometric or radiographic analysis to quantify the number interacting with the selectin polypeptide of solid phase.

One exemplary shear based assay, also referred to as a Stamper-Woodruff assay, is performed as follows. Test cells are placed on a slide by cytocentrifugation and then are incubated with isotype control or with the test antibody (e.g., test anti-CD44 antibody). Cells bearing E-selectin or L-selectin (e.g., CHO-transfected with E-selectin or lymphocytes bearing L-selectin) are then placed over the test cells and rotational shear is applied to the slides. The ability to bind to L-selectin-bearing cells or E-selectin-bearing cells is measured after 30 minutes of shear. A second type of assays uses a parallel plate approach. In this assay, the amount of cells that are in contact with the E-selectin or L-selectin (placed as substrates on the floor of the chamber), the velocity of the cells moving on the substrate (the lower the velocity the better the attachment), and the shear resistance (the level of shear required to achieve detachment) are measured. For measuring the binding to E-selectin or to L-selectin, test cells can be placed as the substrate and cells bearing either E-selectin or L-selectin can be introduced into the chamber under flow conditions. Similarly, the amount of selectin-bearing cells that are in contact with the test cells that have either been incubated with isotype control antibody or with the test antibody are measured. The total number of cells contacting or rolling on the test cells can be measured. The velocity of the selectin-bearing cells on the test cells can be measured. The lower the velocity observed, the better the attachment. Shear resistance (the level of shear required to achieve detachment) can also be measured. Each one of these parameters can be used to identify and/or characterize an anti-CD44 antibody as an agonist or antagonist of a CD44 binding activity. For example, an antagonist can be an antibody that lowers the resistance to detachment at shear than the isotype control, or that renders the cells showing a faster velocity than the isotype, or that lowers the total number of cells in contact with the selectin or with HA. The opposite would be true for an agonist.

Other exemplary assays for determining binding of CD44 to HA include assays that evaluate binding of HA-FITC (which is measured by flow cytometry) and the binding of test cells to HA deposited on plates. The binding to HA-FITC allows for quantitation of a shift in fluorescence intensity measured by flow cytometry of test cells that are either incubated with control isotype antibody or incubated with test antibody before exposure to HA-FITC. The binding to HA on plates can be measured as an absolute number (e.g., the number of test cells adhering to HA before and after incubation with a control or test antibody) or the effect(s) of the antibody on the cells under flow conditions in a parallel plate assay can be measured. Under the latter conditions, the numbers of cells that are in contact with the HA, and the velocity of the cells moving on the HA (the lower the velocity the better the attachment), and shear resistance (the level of shear required to achieve detachment) for cells that were incubated with isotype control or the test antibody, are measured. For measuring the binding to E-selectin or to L-selectin, soluble forms of E-selectin and L-selectin (e.g., E-selectin-Ig and L-selectin-Ig constructs, where these molecules are linked to the Fc portion of human Ig) can be used. The amount of E-selectin or L-selectin bound to a test cell (incubated with isotype-match control or with test mAb) can be measured by flow cytometry using anti-human Ig-FITC reagents to detect the level of binding of the selectin molecule(s).

Derivatized Antibodies

An anti-CD44 antibody, or antigen binding portion thereof, can be derivatized or linked to another functional molecule (e.g., another peptide or protein). Accordingly, the antibodies and antibody portions of the invention are intended to include derivatized and otherwise modified forms of the antibodies described herein, including immunoadhesion molecules. For example, an antibody or antibody portion of the invention can be functionally linked (by chemical coupling, genetic fusion, noncovalent association or otherwise) to one or more other molecular entities, such as another antibody, a detectable agent, a cytotoxic agent, a pharmaceutical agent, and/or a protein or peptide that can mediate association of the antibody or antibody portion with another molecule (such as a magnetic bead, streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an antibody or antibody portion of the invention may be derivatized (or labeled) to include fluorescent compounds, various enzymes, prosthetic groups, luminescent materials, bioluminescent materials, fluorescent emitting metal atoms, e.g., europium (Eu), and other anthanides, and radioactive materials (described below). Exemplary fluorescent detectable agents include fluorescein, fluorescein isothiocyanate, rhodamine, 5-dimethylamine-1-napthalenesulfonyl chloride, phycoerythrin and the like. An antibody may also be derivatized with detectable enzymes, such as alkaline phosphatase, horseradish peroxidase, β-galactosidase, acetylcholinesterase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody may also be derivatized with a prosthetic group (e.g., a magnetic bead, streptavidin/biotin and avidin/biotin). For example, an antibody may be derivatized with biotin, and detected through indirect measurement of avidin or streptavidin binding. Examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of bioluminescent materials include luciferase, luciferin, and aequorin.

Labeled antibodies can be used, for example, diagnostically and/or experimentally in a number of contexts, including (i) to isolate a predetermined antigen by standard techniques, such as affinity chromatography or immunoprecipitation; (ii) to detect a predetermined antigen (e.g., in a cellular lysate or cell supernatant) in order to evaluate the abundance and pattern of expression of the protein; (iii) to monitor protein levels in tissue as part of a clinical testing procedure, e.g., to determine the efficacy of a given treatment regimen.

An anti-CD44 antibody or antigen binding portion thereof may be conjugated to a another molecular entity (an “antibody conjugate”), typically a label or a therapeutic (e.g., a cytotoxic or cytostatic) agent or moiety.

Radioactive isotopes can be used in diagnostic or therapeutic applications. Radioactive isotopes that can be coupled to the anti-CD44 antibodies include, but are not limited to α-, β-, or γ-emitters, or β- and γ-emitters. Such radioactive isotopes include, but are not limited to iodine (¹³¹I or ¹²⁵I), yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), indium (¹¹¹In), technetium (⁹⁹mTc), phosphorus (³²P), rhodium (¹⁸⁸Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), or gallium (⁶⁷Ga). Radioisotopes useful as therapeutic agents include yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), and rhodium (¹⁸⁸Rh). Radioisotopes useful as labels, e.g., for use in diagnostics, include iodine (¹³¹I or ¹²⁵I), indium (¹¹¹In), technetium (⁹⁹mTc), phosphorus (³²P), carbon (¹⁴C), and tritium (³H), or one or more of the therapeutic isotopes listed above.

The invention provides radiolabeled anti-CD44 antibodies and antigen binding portions thereof, and methods of labeling the same. Known methods of labeling an antibody can be used. For example, an anti-CD44 antibody or antigen binding portion thereof can be contacted with a chelating agent, e.g., 1,4,7,10-tetraazacyclododecane-N,N′,N″,N′″-tetraacetic acid (DOTA), to produce a conjugated antibody. The conjugated antibody or antigen binding portion thereof can then be radiolabeled with a radioisotope to thereby produce a labeled anti-CD44 antibody or antigen binding portion thereof.

As is discussed above, the antibody or antigen binding portion thereof can be conjugated to a therapeutic agent. The conjugates of the invention can be used for modifying a given biological response. The therapeutic agent is not to be construed as limited to classical chemical therapeutic agents. For example, the therapeutic agent may be a protein or polypeptide possessing a desired biological activity. Such proteins may include, for example, a toxin such as abrin, ricin A, pseudomonas exotoxin, diphtheria toxin, or a component thereof; a protein such as tumor necrosis factor, interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors. Similarly, the therapeutic agent can be a viral particle, e.g., a recombinant viral particle, that is conjugated (e.g., via a chemical linker) or fused (e.g., via a viral coat protein) to anti-CD44 antibody molecules of the invention.

Nucleic Acids, Vectors and Host Cells

Another aspect of the invention pertains to isolated nucleic acid, vector and host cell compositions that can be used for recombinant expression of the antibodies and antigen binding portion of the invention. In one embodiment, a first and second isolated nucleic acid comprising a nucleotide sequence encoding heavy and light chain variable regions, respectively, of an anti-CD44 antibody, or an antigen binding portion thereof, are provided.

In another aspect, the invention features host cells and vectors (e.g., recombinant expression vectors) containing the nucleic acids, e.g., the first and second nucleic acids, of the invention.

Prokaryotic or eukaryotic host cells may be used. The terms “host cell” and “recombinant host cell” are used interchangeably herein. Such terms refer not only to the particular subject cell, but to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic, e.g., bacterial cells such as E. coli, or eukaryotic, e.g., insect cells, yeast, or preferably mammalian cells (e.g., cultured cell or a cell line). Other suitable host cells are known to those skilled in the art.

Preferred mammalian host cells for expressing the anti-CD44 antibodies, or antigen binding portions thereof, include Chinese Hamster Ovary (CHO cells), lymphocytic cell lines, and COS cells.

In another aspect, the invention features a vector, e.g., a recombinant expression vector. The recombinant expression vectors of the invention can be designed for expression of the antibodies, or an antigen binding portion thereof, in prokaryotic or eukaryotic cells. For example, polypeptides of the invention can be expressed in E. coli, insect cells (e.g., using baculovirus expression vectors), yeast cells or mammalian cells. Suitable host cells are discussed further in Goeddel, (1990) Gene Expression Technology: Methods in Enzymology 185, Academic Press, San Diego, Calif. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to an antibody encoded therein, usually to the constant region of the recombinant antibody.

In addition to the antibody chain genes, the recombinant expression vectors of the invention carry regulatory sequences that are operatively linked and control the expression of the antibody chain genes in a host cell.

Pharmaceutical Compositions

In another aspect, the present invention provides compositions, e.g., pharmaceutically acceptable compositions, which include an antibody molecule described herein, formulated together with a pharmaceutically acceptable carrier.

As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g., by injection or infusion).

The compositions of this invention may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Typical preferred compositions are in the form of injectable or infusible solutions. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions typically should be sterile and stable under the conditions of manufacture and storage. The composition can be formulated as a solution, microemulsion, dispersion, liposome, or other ordered structure suitable to high antibody concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibodies and antibody portions of the present invention can be administered by a variety of methods known in the art. As will be appreciated by the skilled artisan, the route and/or mode of administration will vary depending upon the desired results.

In certain embodiments, an antibody or antibody portion of the invention may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation. Therapeutic compositions can be administered with medical devices known in the art.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of an antibody or antibody portion of the invention is 0.1-20 mg/kg, more preferably 1-10 mg/kg. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the antibody or antibody portion may vary according to factors such as the disease state, age, sex, and weight of the individual, and the ability of the antibody or antibody portion to elicit a desired response in the individual. A therapeutically effective amount is also one in which any toxic or detrimental effects of the antibody or antibody portion is outweighed by the therapeutically beneficial effects. A “therapeutically effective dosage” preferably inhibits a measurable parameter, e.g., tumor growth rate by at least about 20%, more preferably by at least about 40%, even more preferably by at least about 60%, and still more preferably by at least about 80% relative to untreated subjects. The ability of a compound to inhibit a measurable parameter can be evaluated in an animal model system predictive of efficacy in humans. Alternatively, this property of a composition can be evaluated by examining the ability of the compound to modulate, such modulation in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Also within the scope of the invention are kits comprising an anti-CD44 antibody or antigen binding portion thereof. The kit can include one or more other elements including: instructions for use; other reagents, e.g., a label, a therapeutic agent, or an agent useful for chelating, or otherwise coupling, an antibody to a label or therapeutic agent, or a radioprotective composition; devices or other materials for preparing the antibody for administration; pharmaceutically acceptable carriers; and devices or other materials for administration to a subject. Instructions for use can include instructions for diagnostic applications of the anti-CD44 antibodies (or antigen binding portion thereof) to detect CD44, in vitro, e.g., in a sample, e.g., a biopsy or cells from a patient, or in vivo. The instructions can include instructions for therapeutic application including suggested dosages and/or modes of administration, e.g., in a patient with a hematopoietic disorder. Other instructions can include instructions on coupling of the antibody to a chelator, a label or a therapeutic agent, or for purification of a conjugated antibody, e.g., from unreacted conjugation components. As discussed above, the kit can include a label, e.g., any of the labels described herein. As discussed above, the kit can include a therapeutic agent, e.g., a therapeutic agent described herein. The kit can include a reagent useful for chelating or otherwise coupling a label or therapeutic agent to the antibody, e.g., a reagent discussed herein. In some applications, the antibody will be reacted with other components, e.g., a chelator or a label or therapeutic agent, e.g., a radioisotope. In such cases the kit can include one or more of a reaction vessel to carry out the reaction or a separation device, e.g., a chromatographic column, for use in separating the finished product from starting materials or reaction intermediates.

The kit can further contain at least one additional reagent, such as a diagnostic or therapeutic agent, e.g., a diagnostic or therapeutic agent as described herein, and/or one or more additional anti-CD44 antibodies (or portions thereof), formulated as appropriate, in one or more separate pharmaceutical preparations.

### USES OF THE INVENTION

The antibodies and antigen binding portions thereof have in vitro and in vivo diagnostic, therapeutic and prophylactic utilities. For example, the antibodies and antigen binding portions thereof can be used in methods that include the steps of contacting a cell with an anti-CD44 antibody or antigen binding portion thereof (e.g., an anti-CD44 antibody or antigen binding portion thereof described herein), in an amount sufficient to modulate an activity of the cell (e.g., modulate binding to a CD44 ligand such as hyaluronan, E-selectin, or L-selectin) or ablate, or kill the cell. These antibodies and antigen binding portions thereof can be administered to cells in culture, e.g. in vitro or ex vivo, or in a subject, e.g., in vivo, to treat, prevent, and/or diagnose a variety of disorders, such hematopoietic disorders, inflammatory disorders, cancer-related disorders, and autoimmune disorders. For in vitro embodiments, the cells, e.g., hematopoietic cells, can be cultured in vitro in culture medium and the contacting step can be effected by adding the anti-CD44 antibody or portion thereof, to the culture medium. These manipulations include the incubation of cells (such as stem cells) with antibodies directed to CD44/HCELL to enhance growth of the cells ex vivo (expansion) or to render such cells capable of higher efficiency homing in vivo through enhanced capacity to engage CD44/HCELL ligands such as hyaluronic acid, L-selectin, and E-selectin.

For in vivo embodiments, the contacting step is effected in a subject and includes administering the anti-CD44 antibody or portion thereof to the subject under conditions effective to permit both binding of the antibody or portion to the cell, and the treating, e.g., the modulating, killing, or ablating of the cell.

The antibodies and antigen binding portions thereof can be used to purify CD44 (e.g., HCELL) for commercial use.

As used herein, the term “subject” is intended to include human and non-human animals. The term “non-human animals” of the invention includes all vertebrates, e.g., mammals and non-mammals, such as non-human primates, sheep, dog, cow, chickens, amphibians, reptiles, etc.

In one embodiment, the subject is a human subject.

Hematopoietic Applications

The anti-CD44 antibodies and antigen binding portions thereof and methods described herein can be used, e.g., in the purification, enrichment, and characterization of hematopoietic cells. The anti-CD44 antibodies and antigen binding portions thereof and methods of using the antibodies and antigen binding portions thereof can also be used in the treatment of hematopoietic disorders (e.g., aplastic anemia), including hematopoietic cancer related disorders (e.g., in the treatment of hematopoietic cancers, e.g., for hematopoietic stem cell transplantation associated with hematopoietic cancers, and for hematopoietic stem cell transplantation associated with non-hematopoietic cancers), anemia, neutropenia, and other bone marrow failure states.

Methods of detecting CD44-expressing cells can be used, e.g., to characterize a population of hematopoietic cells. Anti-CD44 antibodies and antigen binding portions thereof can be used to isolate CD44-expressing cells, e.g., from blood or bone marrow samples. Anti-CD44 antibodies and antigen binding portions thereof that bind the HCELL glycoform of CD44 can be used to isolate HCELL-expressing cells, such as hematopoietic progenitor cells. Antibodies that bind HCELL (e.g., HCELL present on HCELL-expressing cells) can be used to enrich for HCELL⁺ hematopoietic progenitor cells, e.g., during processing of bone marrow for bone marrow transplantation, thus enriching for a population of cells with a high capacity to home to the bone marrow and form blood cells. Antibodies and antigen binding portions thereof that agonize an HCELL function, such as L-selectin or E-selectin binding, can be used to promote engraftment and/or the blood-forming capacity of hematopoietic progenitor cells in vivo. Additionally, these antibodies may be used to agonize HCELL function to achieve expansion of both non-hematopoietic and hematopoietic stem cells, and can be used to enhance homing and engraftment of adult stem cells to tissues for regenerative therapy.

The anti-CD44 antibodies and antigen binding portions thereof and methods described herein can be used in method of enhancing engraftment of hematopoietic cells in vivo. Antibodies and portions thereof that agonize interaction of HCELL with L-selectin or E-selectin, e.g., can enhance adhesion of hematopoietic progenitor cells to sites of hematopoiesis or to sites of tissue injury (regenerative therapy), thereby promoting engraftment. Enhanced engraftment is desirable in treating any disorder that requires bone marrow ablation and bone marrow transplant, e.g., for treatment of cancers, immunodeficiency disorders, and other hematopoietic disorders, or in tissue injury in non-bone marrow sites such as skin in burns, or cardiac or nervous system tissue following ischemic injury.

Inflammatory and Autoimmune Disorders

Anti-CD44 antibodies and antigen binding portions thereof can be used in methods of treating inflammatory or autoimmune disorders. For example, anti-CD44 antibodies and antigen binding portions thereof that block binding of CD44-expressing leukocytes to CD44 ligands can dampen immune responses by decreasing adhesion of CD44-expressing leukocytes to sites of inflammation. Anti-CD44 antibodies and antigen binding portions thereof can also be used in situations in which augmented immune responses are desirable, e.g., to enhance a response to an infectious agent. For example, anti-CD44 antibodies and antigen binding portions thereof that agonize an activity of CD44, such as CD44-hyaluronon binding interactions, can be used to stimulate adhesion of CD44-expressing leukocytes.

Examples of inflammatory and autoimmune diseases include, but are not limited to: cutaneous inflammatory disorders include dermatitis (e.g., contact dermatitis, atopic dermatitis), cutaneous infection (e.g., bacterial, viral, fungal, or parasitic infections of the skin), pemphigus vulgaris, graft versus host disease (e.g., acute cutaneous graft versus host disease), psoriasis, drug-related hypersensitivity reactions, ultraviolet light induced injury, and acne. Other inflammatory disorders that can be treated according to the methods described herein include pulmonary disorders such as acute lung injury, acute respiratory distress syndrome, asthma, bronchitis, and cystic fibrosis. Inflammatory disorders that can be treated according to methods described herein include inflammatory disorders of joints, such as arthritis (e.g., rheumatoid arthritis); inflammatory disorders of the gut, such as inflammatory bowel disease, Crohn's disease and ulcerative colitis; inflammatory disorders of the nervous system, such as encephalomyelitis, and multiple sclerosis; inflammatory disorders of the endocrine system (e.g., Grave's disease, Hashimoto's thyroiditis); multiorgan inflammatory disorders (e.g., systemic lupus erythematosus); and renal, vascular, and hepatic disorders such as hepatitis (e.g., viral chronic hepatitis), reperfusion injury (e.g., myocardial), nephritis, pancreatitis, artery occlusion (e.g., retinal), and/or vasculitis. The inflammatory disease may be acute or chronic, and is preferably mediated by leukocytes. In some embodiments, the inflammatory disease is associated with chronic inflammation, e.g., the disease is inflammatory bowel disease, such as, psoriasis, sarcoidosis and rheumatoid arthritis.

Cancers

Anti-CD44 antibodies and antigen binding portions thereof can be used, e.g., in methods of treating, preventing, and diagnosing cancers. For example, an anti-CD44 antibody or antigen binding portion thereof can be conjugated to a cytotoxic moiety (e.g., a toxin or a radioisotope) and used to ablate or reduce the growth of CD44-expressing cancer cells. Anti-CD44 antibodies and antigen binding portions thereof can be used to characterize expression of CD44 on various hematopoietic cancers. Anti-CD44 antibodies and antigen binding portions thereof that increase a CD44 activity (e.g., increase binding of CD44 to a CD44 ligand) can be used to augment immune reactivity to cancer cells, e.g., in method of immunotherapy of cancers.

As used herein, the term “cancer” is meant to include all types of cancerous growths or oncogenic processes, metastatic tissues or malignantly transformed cells, tissues, or organs, irrespective of histopathologic type or stage of invasiveness. Examples of cancers that can be treated or prevented include, but are not limited to, CD44-expressing cancers (e.g., HCELL-expressing cancers, e.g., HCELL-expressing acute lymphocytic leukemia, or HCELL-expressing acute myeloid leukemia). Other cancers that can be treated or prevented include cutaneous cancers such as melanomas, basal cell and squamous cell carcinomas, Kaposi's sarcoma, leukemias, and lymphomas (e.g., chronic myeloid leukemia, chronic lymphatic leukemia, chronic granulocytic leukemia, acute lymphocytic leukemia, acute myeloid leukemia, acute myelocytic leukemia, Hodgkin's disease, non-hodgkin's lymphomas, Burkitt's lymphomas, and mycosis fungoides). Also included are neoplastic disorders and disorders of solid tumors such as adenocarcinomas of the breast, lung, kidney, uterus, prostate, bladder, ovary, colon; sarcomas such as liposarcoma, synovial sarcoma, rhabdomyosarcoma, Ewing's tumor, neuroepithelioma; and other tumors such as glioblastoma, retinoblastoma, Wilm's tumor, and mesothelioma. The most preferred cancers are breast, ovarian, colon and melanoma, where in each case CD44 interactions with hyaluronic acid plays a role in growth and metastasis.

Contraception and Fertility Disorders

Anti-CD44 antibodies and antigen binding portions thereof can be used in methods of contraception. For example, antibodies that block L-selectin binding by HCELL can be used to inhibit selectin-dependent (e.g., L-selectin-dependent) interactions that occur during implantation of an embryo. Anti-CD44 antibodies and antigen binding portions thereof can also be used to promote pregnancy. For example, antibodies that agonize interaction of HCELL with selectins (e.g., L-selectin) can be used to stimulate implantation of an embryo.

Methods of administering antibody molecules and portions thereof are described above. Suitable dosages of the molecules used will depend on the age and weight of the subject and the particular drug used. The antibody molecules can be used as competitive agents for ligand binding to inhibit, reduce an undesirable interaction, or to increase a desirable interaction.

In one embodiment, the anti-CD44 antibodies or antigen binding portions thereof can be used to kill or ablate cells in vivo. For example, the anti-CD44 antibodies or portions thereof can be used to treat or prevent a disorder described herein. The antibodies (or portions thereof) can be used by themselves or conjugated to a second agent, e.g., a cytotoxic drug, radioisotope, or a protein, e.g., a protein toxin or a viral protein. This method includes: administering the antibody or portion thereof, alone or conjugated to a cytotoxic drug, to a subject requiring such treatment.

The antibodies and antigen binding portions thereof of the present invention may be used to deliver a variety of therapeutic agents, e.g., a cytotoxic moiety, e.g., a therapeutic drug, a radioisotope, molecules of plant, fungal, or bacterial origin, or biological proteins (e.g., protein toxins) or particles (e.g., a recombinant viral particles, e.g., via a viral coat protein), or mixtures thereof. The therapeutic agent can be an intracellularly active drug or other agent, such as short-range radiation emitters, including, for example, short-range, high-energy α-emitters, as described herein. In some preferred embodiments, the anti-CD44 antibody or antigen binding portion thereof can be coupled to a molecule of plant or bacterial origin (or derivative thereof).

Combination Therapy

The anti-CD44 antibodies and antigen binding portions thereof described herein may be used in combination with other therapies. For example, the combination therapy can include a composition of the present invention coformulated with, and/or coadministered with, one or more additional therapeutic agents, e.g., one or more anti-cancer agents, cytotoxic or cytostatic agents, anti-inflammatory agents, immunosuppressive agents, hormone treatment, vaccines, and/or other therapies. Anti-inflammatory agents that can be administered in combination with anti-CD44 antibodies and antigen binding portions thereof include non-steroidal anti-inflammatory drugs (e.g., aspirin, ibuprofen, naproxen, and the like), COX-2 inhibitors (e.g., rofecoxib, and celecoxib); leukotriene synthesis inhibitors, leukotriene receptor antagonists, glucocorticosteroids, steroid chemical derivatives, proinflammatory cytokine antagonists, and antibodies to molecules involved in the regulation of immune responses (e.g., anti-CD3 antibodies, anti-CD4 antibodies). Immunosuppressive agents that can be administered in combination with anti-CD44 antibodies and antigen binding portions thereof include cyclosporine, FK-506, dexamethasone, and azathioprine. In other embodiments, the anti-CD44 antibodies and antigen binding portions thereof are administered in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications associated with the various monotherapies.

Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated. In other embodiments, one or more of the treatments are delivered to a subject at risk for a disorder, and, e.g., one or more additional treatments can be administered while the subject is still at risk, e.g., to a transplant recipient. In some embodiments, the delivery of one treatment is still occurring when the delivery of the second begins, so that there is overlap. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

Diagnostic Uses

In one aspect, the present invention provides a diagnostic method for detecting the presence of a CD44 protein in vitro (e.g., in a biological sample, such as a tissue biopsy, e.g., from bone marrow) or in vivo (e.g., in vivo imaging in a subject). The method includes: (i) contacting the sample with an anti-CD44 antibody or portion thereof, or administering to the subject, the anti-CD44 antibody or portion thereof; (optionally) (ii) contacting a reference sample, e.g., a control sample (e.g., a control biological sample, such as plasma, tissue, biopsy) or a control subject)); and (iii) detecting formation of a complex between the anti-CD44 antibody or portion thereof, and the sample or subject, or the control sample or subject, wherein a change, e.g., a statistically significant change, in the formation of the complex in the sample or subject relative to the control sample or subject is indicative of the presence of CD44, e.g., HCELL, in the sample.

Preferably, the anti-CD44 antibody (or portion thereof) is directly or indirectly labeled with a detectable substance to facilitate detection of the bound or unbound antibody. Suitable detectable substances include various enzymes, prosthetic groups, fluorescent materials, luminescent materials and radioactive materials, as described above and described in more detail below.

Complex formation between the anti-CD44 antibody or portion thereof and CD44 can be detected by measuring or visualizing either the antibody (or antibody portion) bound to the CD44 antigen or unbound antibody (or antibody portion). Conventional detection assays can be used, e.g., an enzyme-linked immunosorbent assays (ELISA), a radioimmunoassay (RIA) or tissue immunohistochemistry. Alternative to labeling the anti-CD44 antibody or portion thereof, the presence of CD44 can be assayed in a sample by a competition immunoassay utilizing standards labeled with a detectable substance and an unlabeled anti-CD44 antibody or portion thereof. In this assay, the biological sample, the labeled standards and the CD44 binding agent are combined and the amount of labeled standard bound to the unlabeled antibody is determined. The amount of CD44 in the sample is inversely proportional to the amount of labeled standard bound to the CD44 binding agent.

### EXAMPLE

To raise monoclonal antibodies against HCELL, purified HCELL obtained from KG1a-RS cells (a subline of KG1a cells) was injected into Balb/c mice and monoclonal antibodies were developed by standard methods. These mAb were screened against KG1a-RS cells by flow cytometry (FACS). Monoclonal antibodies (mAb) reactive agains KG1a-RS cells were then expanded and the capability of the mAb to recognize CD44 from other hematopoietic cell lines and from normal hematopoietic cells was measured by FACS. All mAb that reacted to KG1a-RS and that detected CD44 by Western blot analysis were subdivided into groups depending on their pattern of reactivity with hematopoietic cell lines and normal blood cells and whether they have functional capabilities. A number of mAb that were raised identify only the HCELL glycoform of CD44 (HCELL-specific mAb), but many recognize CD44 without HCELL-specific modifications. All mAb raised were tested to determine whether they (1) inhibit the function of HCELL as an E-selectin or an L-selectin ligand, (2) potentiate the function of HCELL as an E-selectin or an L-selectin ligand, (3) inhibit the binding of HCELL to hyaluronic acid (HA), or (4) potentiate binding of CD44 to hyaluronic acid. Several mAb have been identified which affect HA binding and also affect selectin ligand activity. mAbs were isolated which (1) have the capacity to enhance or inhibit the selectin and HA binding functions of HCELL (function-blocking and function-enhancing mAb), and (2) that recognize only subsets of human hematopoietic stem cells that express the HCELL glycoform of CD44 (HCELL-specific mAb).

### EQUIVALENTS

Those skilled in the art will recognize, or be able to ascertain using no more than routine experimentation, many equivalents of the specific embodiments of the invention described herein. Such equivalents are intended to be encompassed by the following claims.

1. An isolated antibody or antigen binding portion thereof that binds to a CD44 glycoprotein, wherein the antibody or antigen binding portion thereof comprises one or more of the following characteristics:
(a) the antibody or antigen binding portion thereof modulates binding of CD44 to hyaluronan;
(b) the antibody or antigen binding portion thereof modulates binding of HCELL to E-selectin;
(c) the antibody or antigen binding portion thereof modulates binding of HCELL to L-selectin.

2. The antibody or antigen binding portion thereof of claim 1, wherein the antibody or antigen binding portion thereof comprises characteristic (a), and wherein the antibody or antigen binding portion thereof agonizes binding of CD44 to hyaluronan.

3. The antibody or antigen binding portion thereof of claim 1, wherein the antibody or antigen binding portion thereof comprises characteristic (a), wherein the antibody or antigen binding portion thereof inhibits binding of CD44 to hyaluronan.

4. The antibody or antigen binding portion thereof of claim 1, wherein the antibody or antigen binding portion thereof comprises characteristic (b), and wherein the antibody or antigen binding portion thereof agonizes binding of HCELL to E-selectin.

5. The antibody or antigen binding portion thereof of claim 1, wherein the antibody or antigen binding portion thereof comprises characteristic (b), and wherein the antibody or antigen binding portion thereof inhibits binding of HCELL to E-selectin.

6. The antibody or antigen binding portion thereof of claim 1, wherein the antibody or antigen binding portion thereof comprises characteristic (c), and wherein the antibody or antigen binding portion thereof agonizes binding of HCELL to L-selectin.

7. The antibody or antigen binding portion thereof of claim 1, wherein the antibody or antigen binding portion thereof comprises characteristic (c), and wherein the antibody or antigen binding portion thereof inhibits binding of HCELL to L-selectin.

8. The antibody or antigen binding portion thereof of claim 1, wherein the antibody or antigen binding portion thereof comprises characteristics (a) and (b).

9. The antibody or antigen binding portion thereof of claim 8, wherein the antibody or antigen binding portion thereof agonizes binding of CD44 to hyaluronan and binding of HCELL to E-selectin.

10. The antibody or antigen binding portion thereof of claim 8, wherein the antibody or antigen binding portion thereof agonizes binding of CD44 to hyaluronan and inhibits binding of HCELL to E-selectin.

11. The antibody or antigen binding portion thereof of claim 8, wherein the antibody or antigen binding portion thereof inhibits binding of CD44 to hyaluronan and inhibits binding of HCELL to E-selectin.

12. The antibody or antigen binding portion thereof of claim 8, wherein the antibody or antigen binding portion thereof inhibits binding of CD44 to hyaluronan and agonizes binding of HCELL to E-selectin.

13. The antibody or antigen binding portion thereof of claim 8, wherein the antibody or antigen binding portion thereof does not specifically bind to a non-HCELL glycoform of CD44.

14. The antibody or antigen binding portion thereof of claim 1, wherein the antibody or antigen binding portion thereof comprises characteristics (a) and (c).

15. The antibody or antigen binding portion thereof of claim 14, wherein the antibody or antigen binding portion thereof does not specifically bind to a non-HCELL glycoform of CD44.

16. The antibody or antigen binding portion thereof of claim 1, wherein the antibody or antigen binding portion thereof comprises characteristics (b) and (c).

17. The antibody or antigen binding portion thereof of claim 16, wherein the antibody or antigen binding portion thereof does not specifically bind to a non-HCELL glycoform of CD44.

18. The antibody or antigen binding portion thereof of claim 1, wherein the antibody or antigen binding portion thereof comprises characteristics (a), (b), and (c).

19. The antibody or antigen binding portion thereof of claim 2, wherein the antibody or antigen binding portion thereof does not specifically bind to a non-HCELL glycoform of CD44.

20. The antibody or antigen binding portion thereof of claim 1, wherein the antibody or antigen binding portion thereof comprises one or more of the following characteristics:
(d) the antibody or antigen binding portion thereof does not compete for binding to an epitope bound by HECA-452 on CD44;
(e) the antibody or antigen binding portion thereof competes for binding to an epitope bound by HECA-452;
(f) the antibody or antigen binding portion thereof binds an epitope that overlaps with a Sialyl Lewis X/A determinant on CD44.

21. The antibody or antigen binding portion thereof of claim 1, wherein the antibody or antigen binding portion binds a saccharide epitope of CD44.

22. The antibody or antigen binding portion thereof of claim 21, wherein the antibody or antigen binding portion thereof binds a selectin-binding saccharide epitope of CD44.

23. The antibody or antigen binding portion thereof of claim 1, wherein the antibody or antigen binding portion thereof binds a protein epitope of CD44.

24. The antibody or antigen binding portion thereof or claim 1, wherein the antibody or antigen binding portion thereof binds a protein and a saccharide epitope of CD44.

25. An isolated antibody or antigen binding portion thereof that specifically binds to an HCELL glycoprotein, wherein the antibody or antigen binding portion thereof binds a denatured form of HCELL.

26. An isolated antibody or antigen binding portion thereof that specifically binds to CD44, wherein the antibody is made by a method which comprises:
purifying HCELL from a KG1acell;
injecting the purified HCELL into a mouse;
obtaining a monoclonal antibody from the mouse which binds HCELL.

27. An isolated antibody or antigen binding portion thereof that specifically binds to an HCELL glycoprotein, wherein the antibody or antigen binding portion thereof enhances engraftment of hematopoietic stem cells in vivo.

28. The antibody or antigen binding portion thereof of claim 1, wherein the antibody specifically binds a human CD44 or HCELL glycoprotein.

29. The antibody or antigen binding portion thereof of claim 1, wherein the antibody is a monoclonal or polyclonal antibody.

30. The antibody or antigen binding portion thereof of claim 1, wherein the antibody is humanized.

31. The antibody or antigen binding portion thereof of claim 1, wherein the antibody is chimeric, deimmunized, or human.

32. A pharmaceutical composition comprising an antibody or antigen binding portion thereof of claim 1, and a pharmaceutically acceptable carrier.

33. A method of modulating an activity of an HCELL-expressing cell, the method comprising: contacting an HCELL-expressing cell with an antibody or antigen binding portion thereof of claim 1, thereby modulating the activity of the HCELL-expressing cell.

34. A method of modulating HCELL activity in a subject, the method comprising:
identifying a subject in need of modulated HCELL activity; and administering the antibody or antigen binding portion thereof of claim 1 to the subject.

35. A method of modulating HCELL activity in a subject, the method comprising:
identifying a subject in need of modulated HCELL activity;
providing a population of cells from the subject;
contacting the cells with an antibody or antigen binding portion thereof of claim 1 under conditions that allow the antibody or antigen binding portion thereof to bind HCELL-expressing cells in the population;
administering the cells to the subject.

36. A method for treating a disorder characterized by HCELL-selectin interactions, the method comprising:
administering to a subject an antibody or antigen binding portion thereof that modulates an HCELL-selectin interaction, to thereby treat the disorder.

37. The method of claim 36, wherein the disorder is a hematopoietic disorder, an inflammatory or autoimmune disorder, a vascular disease or a cancer-related disorder.

38. A method for isolating a subset of hematopoietic cells, the method comprising:
providing a population of cells, wherein the population comprises hematopoietic cells;
contacting the cells with an anti-HCELL antibody or antigen binding portion thereof under conditions that allow the anti-HCELL antibody or antigen binding portion thereof to bind the HCELL-expressing cells;
removing the cells that do not bind the anti-HCELL antibody or antigen binding portion thereof, thereby isolating a subset of hematopoietic cells.

39. A method for enriching a population of hematopoietic cells for cells that have blood-forming capacity, the method comprising:
providing a population of hematopoietic cells;
contacting the cells with an anti-HCELL antibody or antigen binding portion thereof under conditions that allow the anti-HCELL antibody or antigen binding portion thereof to bind the HCELL-expressing cells;
removing the cells that do not bind the anti-HCELL antibody or antigen binding portion thereof, thereby enriching a population of hematopoietic cells for cells that have blood-forming capacity.

40. A method for enhancing engraftment of hematopoietic stem cells in a subject, the method comprising:
administering to the subject an anti-HCELL antibody or antigen binding portion thereof, wherein the antibody or antigen binding portion thereof agonizes HCELL-selectin interactions, thereby enhancing engraftment of hematopoietic stem cells in the subject.

41. A method for characterizing a hematopoietic cancer, the method comprising: evaluating binding of an anti-HCELL antibody to the cancer, thereby characterizing the cancer.

42. A method for treating a CD44-expressing cancer in a subject, the method comprising:
administering to a subject an anti-CD44 antibody or antigen binding portion thereof, to thereby treat the cancer.

43. A method for increasing an immune response to a cancer in a subject, the method comprising:
administering to a subject an anti-CD44 antibody or antigen binding portion thereof, to thereby increase the immune response to the cancer.

44. A method for treating a hematopoietic cancer in a subject, the method comprising:
administering to a subject an anti-HCELL antibody or antigen binding portion thereof, to thereby treat the cancer.

45. A method for treating an inflammatory or autoimmune disorder or vascular disease, the method comprising:
administering to a subject an anti-CD44 antibody or antigen binding portion thereof, to thereby treat the disorder.

46. A method for treating an inflammatory or autoimmune disorder, the method comprising:
administering to a subject an anti-HCELL antibody or antigen binding portion thereof, to thereby treat the disorder.

47. A method for treating an infectious disorder, the method comprising:
administering to a subject an anti-HCELL antibody, or antigen binding portion thereof, to thereby treat the disorder.

48. A kit comprising:
an antibody that modulates an HCELL-selectin interaction or antigen-binding portion thereof; and
instructions for administering the antibody to a subject at risk for a disorder characterized by HCELL-selectin interactions.

49. A kit comprising:
an antibody that modulates an HCELL-selectin interaction or antigen-binding portion thereof; and
instructions for use of the antibody or antigen binding portion thereof in a method, the method comprising providing a population of cells from a subject;
contacting the cells with an antibody or antigen binding portion thereof of claim 1 under conditions that allow the antibody or antigen binding portion thereof to bind HCELL-expressing cells in the population;
administering the cells to the subject.

50. A kit comprising an antibody or antigen binding portion thereof that specifically binds to an HCELL glycoprotein, wherein the antibody or antigen binding portion thereof comprises at least one of the following characteristics:
(a) the antibody or antigen binding portion thereof binds CD44;
(b) the antibody or antigen binding portion thereof modulates binding of CD44 to hyaluronan;
(c) the antibody or antigen binding portion thereof modulates binding of HCELL to E-selectin;
(d) the antibody or antigen binding portion thereof modulates binding of HCELL to L-selectin.
